(19) United States
(12) Patent Application Publication
(10) Pub. No.: US 2008/0044392 A1
(43) Pub. Date: Feb. 21, 2008

(54) Isolation of Stem Cell-Like Cells and Use Thereof

(75) Inventors: Wilfried A Kues, Neustadt (DE); Heiner Niemann, Neustadt (DE)

Correspondence Address: BROWDY AND NEIMARK, P.L.L.C.;624 NINTH STREET, NW, SUITE 300, WASHINGTON, DC, 20001-5303, US

(73) Assignee: INNOVATIVE DAIRY PRODUCTS PTY LTD AS TRUSTEE FOR THE PARTICIPANTS OF THE COOOPERATIVE RESEARCH CTR, Melbourne VIC (AU)

(21) Appl. No.: 10/576,208

(22) PCT Filed: Oct. 15, 2004
(86) PCT No.: PCT/AU04/01408

§ 371 Date: Mar. 16, 2007

(30) Foreign Application Priority Data
Oct. 17, 2003 (AU) 2003905692

(51) Int. Cl. A61K 35/12; A61P 1/00; A61P 25/00; A61P 3/00; A61P 31/00; A61P 35/00; A61P 37/00; A61P 43/00; A61P 9/00; C12N 15/00; C12N 15/87; C12N 5/00; C12N 5/02; C12N 5/06; C12N 5/08

(52) U.S. Cl. 424/93.21; 424/93.7; 435/325; 435/352; 435/363; 435/366; 435/373; 435/377; 435/378; 435/440; 435/455; 800/21; 800/24

(57) ABSTRACT

The present invention relates to isolated stem cell-like cells and a method of isolation. The invention also relates to a media composition for producing primary cell cultures comprising predominantly tissue-specific progenitor cells or stem cell-like cells. In particular, the present invention relates to an isolated mesenchymal connective tissue-derived stem cell.

### FIELD OF THE INVENTION

The present invention relates to isolated stem cell-like cells and a method of isolation. The invention also relates to a media composition for producing primary cell cultures comprising predominantly tissue-specific progenitor cells or stem cell-like cells. In particular, the present invention relates to a method for the isolation and the selective expansion of mesenchymal connective tissue derived stem cell-like cells (MCTs) from tissue biopsies of fetal and adult donors. The present invention further relates to the use of these cells in somatic nuclear transfer and/or cell therapy.

### BACKGROUND OF THE INVENTION

The advent of stem cell technology has provided a number of exciting new possibilities. For example, the ability to generate tissues or organs from individuals own cells is one step closer. The ability to generate transplant tissues that have been genetically altered so that the recipient immune system does not recognise them as foreign is also closer. This could ultimately lead to xenotransplantation without the associated risks of infection and/or tissue rejection. Finally, improved gene therapy and nuclear transfer techniques can also be developed.

Individuals own stem cells can be genetically altered in vitro, then reintroduced in vivo to produce a desired gene product. These genetically altered stem cells would have the potential to be induced to differentiate to form a multitude of cell types for implantation at specific sites in the body, or for systemic application. Alternately, heterologous stem cells could be genetically altered to express the recipient's major histocompatibility complex (MHC) antigen, or no MHC antigen, allowing transplantation of cells from donor to recipient without the associated risk of rejection.

In the area of nuclear transfer, stem cells are set to make dramatic improvements. For example, standard nuclear transfer techniques typically produce low rates of viable offspring, usually in the range of 0.5-3% of the reconstructed embryos. The efficiency of nuclear transfer techniques has been shown to be partly dependent on the source of donor cells or nuclei. Until the late 1990s it was widely believed that only embryonic or undifferentiated cells or cell nuclei could direct any sort of fetal development in cloning. However, in 1997 Wilmut and co-workers reported successful nuclear transfer experiments using donor cells and nuclei isolated from cultured cell lines (See, e.g., Wilmut et al., Nature (London) 385, 810-183) (1997).

Recently, it has been demonstrated that nuclei of murine embryonic stem cells are significantly more effective in nuclear transfer with regard to viable offspring per NT-blastocyst than somatic fibroblast and cumulus cells, or terminally differentiated blood cells (30-50% vs. 1-3% vs. <0.03% live cloned offspring) (See, for example, Jaenisch et al., 2002, Cloning Stem Cells, 4:389-396 and Hochedlinger & Jaenisch, 2002, Nature, 415:1035-1038.)

Stem cells are defined as cells that have extensive proliferation potential, differentiate into several cell lineages, and repopulate tissues upon transplantation. The quintessential stem cell is the embryonic stem (ES) cell, as it has unlimited self-renewal and multipotent differentiation potential (Orkin, 1998, Int. J. Dev. Biol. 42:927-34; Reubinoff et al., 2000, Nat Biotech, 18:399404; Shamblott et al., 1998, Proc. Natl. Acad. Sci. U.S.A. 95:13726-31; Thomson et al., 1998, Science, 282:114-7; Thomson et al., 1995, Proc. Natl. Acad. Sci. USA. 92:7844-8; Williams et al., 1988, Nature, 336:684-7). These cells are derived from the inner cell mass of the blastocyst or can be derived from the primordial germ cells from a post-implantation embryo (embryonal germ cells or EG cells).

However, while the ES cell has shown the most promise, the supply of these cells is limited in many jurisdictions as the harvesting of ES stem cells necessitates the destruction of the embryo. Therefore, alternative sources of stem cells such as adult stem cells have been sought.

Adult stem cells are a class of cells with apparently pluripotent features in that they appear to have retained their ability to differentiate into other cell types. However, while adult stem cells might be a better alternative source of stem cells than ES cells they are not readily obtained. One of the problems is that adult stem cells are relatively slow growing in vitro. Therefore, when adult stem cells are cultured in a mixed population of cells, the adult stem cells are quickly overgrown by other cells present.

Consequently, it would useful to isolate and proliferate a species of tissue-specific progenitor cells or stem cell-like cells and use these in a number of procedures including nuclear transfer, targeted differentiation and therapeutic treatments.

### SUMMARY OF THE INVENTION

The inventors have now surprisingly found a reliable and selective enrichment process, which is capable of producing primary cell cultures comprising predominantly tissue-specific progenitor cells or stem cell-like cells. More importantly, the inventors have also identified unknown mesenchymal connective tissue-derived stem cells (MCTs), within the primary cell cultures.

Accordingly, a first aspect provides a method for selective culturing of primary cell cultures comprising culturing tissue biopsies in the presence of at least 25% serum relative to the amount of culture medium. Preferably, the serum is between about 25% to about 70%. More preferably, the serum is between about 30% to about 50%. Most preferably, the serum is about 30%.

In one embodiment there is provided a tissue-culture media composition for the selective culturing of primary cell cultures comprising about 30% serum and about 70% culture medium. Preferably, the culture medium is standard tissue culture medium. More preferably, the culture medium is selected from the group consisting of Synthetic Oviductal Fluid (SOF), Modified Eagle's Medium (MEM), Dulbecco's Modified Eagle's Medium (DMEM), RPMI 1640, F-12, IMDM, Alpha Medium and McCoy's Medium. Most preferably, the culture medium is DMEM.

The serum in the culture medium may be allogeneic serum (i.e., from the same animal species, but not the same animal), autologous serum (i.e., from the same animal) or xenogeneic serum (i.e., from a different animal species). Preferably, heat-inactivated autologous serum is used rather than other serum.

While the culture medium may simply be a commercially available medium like DMEM, supplemented with at least 30% serum, it is appreciated that other supplements may be included. For example, growth factors, co-factors, salts and antibiotics may be included.

In one embodiment, about 50% of the culture medium plus serum are replaced about every 48 hours with fresh medium. Accordingly, in a second aspect of the present invention there is provided a method for selective culturing of primary cell cultures comprising:
(i) obtaining a tissue biopsy from an animal;
(ii) culturing said tissue biopsy in tissue culture medium comprising at least 25% serum; and
(iii) replacing about 50% of the culture medium including serum about every 48 hours.

In another embodiment, the tissue biopsies are cultured in the presence of a feeder cell layer. Preferably, the feeder cell layer comprises cultured autologous cells.

A third aspect of the present invention provides an isolated tissue-specific progenitor cell or stem cell-like cell obtained by a method according to the first aspect.

Preferably, the tissue-specific progenitor cell or stem cell-like cell is a mesenchymal connective tissue-derived stem cell (MCT). More preferably, the tissue-specific progenitor cell or stem cell-like cell is the mesenchymal connective tissue-derived stem cell (MCT) deposited under the Budapest Treaty at the Deutsche Sammlung Von Mikroorganismen und Zellkulturen GmbH (DSMZ), Germany on September 2004, under accession number #12345.

The tissue biopsies can be obtained from any animal, including humans. Preferably, the animal is a mammal from the one of the mammalian orders. The mammalian orders include Monotremata, Metatheria, Didelphimorphia, Paucituberculata, Microbiotheria, Dasyuromorphia, Peraamelemorphia, Notoryctemorphia, Diprotodontia, Insectivora, Macroscelidea, Scandentia, Dermoptera, Chiroptera, Primates, Xenarthra, Pholidota, Lagomorpha, Rodentia, Cetacea, Carnivora, Tubulidentata, Proboscidea, Hyracoidea, Sirenia, Perissodactyla and Artiodactyla.

Preferably, the mammal is selected from the group consisting of platypus, echidna, kangaroo, wallaby, shrews, moles, hedgehogs, tree shrews, elephant shrews, bats, primates (including chimpanzees, gorillas, orangutans, humans), edentates, sloths, armadillos, anteaters, pangolins, rabbits, picas, rodents, whales, dolphins, porpoises, carnivores, aardvark, elephants, hyraxes, dugongs, manatees, horses, rhinos, tapirs, antelope, giraffe, cows or bulls, bison, buffalo, sheep, big-horn sheep, horses, ponies, donkeys, mule, deer, elk, caribou, goat, water buffalo, camels, llama, alpaca, pigs and hippos.

In one embodiment, the tissue biopsies are isolated from an ungulate selected from the group consisting of domestic or wild bovid, ovid, cervid, suid, equid and camelid.

Especially preferred ungulates are Bos taurus, Bos indicus, and Bos buffalo cows or bulls.

In another embodiment, the tissue biopsies are isolated from a human subject.

The tissue biopsies may be obtained from different organs, e.g., skin, lung, pancreas, liver, stomach, intestine, heart, reproductive organs, bladder, kidney, urethra and other urinary organs, etc.

Furthermore, the tissue biopsies may be obtained from both fetal and adult tissue.

Once obtained the MCTs of the present invention may be used in any technique that uses stem cells. For example, the MCTs can be used in a method of creating a normal non-human animal; or a method for differentiating the MCTs ex vivo to obtain a cell, tissue or organ, or a method of treating a disease; or a method of cloning a non-human animal.

Accordingly, in a fourth aspect, the present invention provides a method of creating a normal non-human animal comprising the steps of: (a) introducing a MCT into a blastocyst; (b) implanting the blastocyst of (a) into a surrogate mother; and (c) allowing the pups to develop and be born.

Preferably, the animal is chimeric.

In a fifth aspect, the present invention provides a composition comprising a population of MCTs and a culture medium, wherein the culture medium expands the MCTs.

Preferably, the culture medium comprises epidermal growth factor (EGF) and platelet derived growth factor (PDGF). More preferably, the culture medium further comprises leukemia inhibitory factor (LIF).

In a sixth aspect, the present invention provides a composition comprising a population of fully or partially purified MCTs progeny.

Preferably, the progeny have the capacity to be further differentiated. More preferably, the progeny have the capacity to terminally differentiate. Most preferably, the progeny are of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

In a seventh aspect, the present invention provides a method for isolating and propagating MCTs comprising the steps of: (a) obtaining tissue from a mammal; (b) establishing a population of adherent cells; (c) recovering said MCT cells; and (d) culturing MCT cells under expansion conditions to produce an expanded cell population.

In an eighth aspect, the present invention provides an expanded cell population obtained by the method of the seventh aspect.

In a ninth aspect, the present invention provides a method for differentiating MCTs ex vivo comprising the steps of (a) obtaining tissue from a mammal; (b) establishing a population of adherent cells; (c) recovering said MCT cells; (d) culturing MCT cells under expansion conditions to produce an expanded cell population and further comprising (e) culturing the propagated cells in the presence of desired differentiation factors.

Preferably, the differentiation factors are selected from the group consisting of basic fibroblast growth factor (bFGF); vascular endothelial growth factor (VEGF); dimethylsulfoxide (DMSO) and isoproterenol; and, fibroblast growth factor4 (FGF4) and hepatocyte growth factor (HGF).

Preferably, the differentiated cell obtained by the method of aspect nine is ectoderm, mesoderm or endoderm. More preferably, the differentiated cell is of the osteoblast, chondrocyte, adipocyte, fibroblast, marrow stroma, skeletal muscle, smooth muscle, cardiac muscle, occular, endothelial,-epithelial, hepatic, pancreatic, hematopoietic, glial, neuronal or oligodendrocyte cell type.

In a tenth aspect, the present invention provides a method for differentiating MCT cells in vivo comprising the steps of (a) obtaining tissue from a mammal; (b) establishing a population of adherent cells; (c) recovering said MCT cells; (d) culturing MCT cells under expansion conditions to produce an expanded cell population and further comprising (e) administering the expanded cell population to a mammalian host, wherein said cell population is engrafted and differentiated in vivo in tissue specific cells, such that the function of a cell or organ, defective due to injury, genetic disease, acquired disease or iatrogenic treatments, is augmented, reconstituted or provided for the first time.

Preferably, the tissue specific cells are of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

Preferably, the disease is selected from the group consisting of cancer, cardiovascular disease, metabolic disease, liver disease, diabetes, hepatitis, hemophilia, degenerative or traumatic neurological conditions, autoimmune disease, genetic deficiency, connective tissue disorders, anemia, infectious disease and transplant rejection.

In a eleventh aspect, the present invention provides a therapeutic composition comprising MCT cells and a pharmaceutically acceptable carrier, wherein the MCT cells are present in an amount effective to produce tissue selected from the group consisting of bone marrow, blood, spleen, liver, lung, intestinal tract, eye, brain, immune system, bone, connective tissue, muscle, heart, blood vessels, pancreas, central nervous system, kidney, bladder, skin, epithelial appendages, breast-mammary glands, fat tissue, and mucosal surfaces including oral esophageal, vaginal and anal.

In a twelfth aspect, the present invention provides a therapeutic method for restoring organ, tissue or cellular function to a mammalian animal in need thereof comprising the steps of: (a) removing MCT cells from a mammalian donor; (b) expanding MCT cells to form an expanded population of undifferentiated cells; and (c) administering the expanded cells to the mammalian animal, wherein organ, tissue or cellular function is restored.

A thirteenth aspect provides a method of nuclear transfer comprising the step of transferring a mesenchymal connective tissue-derived stem cell or nuclei isolated from a mesenchymal connective tissue-derived stem cell into an enucleated oocyte.

A fourteenth aspect provides a method for producing a genetically engineered or transgenic non-human mammal comprising:
(i) inserting, removing or modifying a desired gene in a mesenchymal connective tissue-derived stem cell (MCT) from a non-human mammal or nuclei isolated from a mesenchymal connective tissue-derived stem cell isolated from a non-human mammal; and
(ii) transferring the MCT or nuclei into an enucleated oocyte.

The invention further provides a method for producing a genetically engineered or transgenic non-human mammal comprising:
(i) inserting, removing or modifying a desired gene or genes in a mesenchymal connective tissue-derived stem cell (MCT) from a non-human mammal or nuclei isolated from a mesenchymal connective tissue-derived stem cell isolated from a non-human mammal; and
(ii) inserting MCT or nuclei into an enucleated oocyte under conditions suitable for the formation of a reconstituted cell;
(iii) activating the reconstituted cell to form an embryo;
(iv) culturing said embryo until greater than the 2-cell developmental stage; and
(v) transferring said cultured embryo to a host mammal such that the embryo develops into a transgenic fetus.

A fifteenth aspect provides a method for cloning a non-human mammal comprising:
(i) inserting a mesenchymal connective tissue-derived stem cell (MCT) from a non-human mammal or nuclei isolated from a mesenchymal connective tissue-derived stem cell isolated from a non-human mammal into an enucleated mammalian oocyte, under conditions suitable for the formation of a reconstituted cell;
(ii) activating the reconstituted cell to form an embryo;
(iii) culturing said embryo until greater than the 2-cell developmental stage; and
(iv) transferring said Cultured embryo to a host mammal such that the embryo develops into a fetus.

Oocytes may be isolated from any mammal by known procedures. For example, oocytes can be isolated from either oviducts and/or ovaries of live animals by oviductal recovery procedures or transvaginal oocyte recovery procedures well known in the art and described herein. Furthermore, oocytes can be isolated from deceased animals. For example, ovaries can be obtained from abattoirs and the oocytes aspirated from these ovaries. The oocytes can also be isolated from the ovaries of a recently sacrificed animal or when the ovary has been frozen and/or thawed. Preferably, the oocytes are freshly isolated from the oviducts.

Oocytes or cytoplasts may also be cryopreserved before use.

In one embodiment, the enucleated oocyte is a zona pellucida-free oocyte. Removal of the zona pellucida can be accomplished by any known procedure. Preferably, the step of removing the zona pellucida is selected from the group consisting of physical manipulation, chemical treatment and enzymatic digestion. More preferably, the zona pellucida is removed by enzymatic digestion. Preferably, the enzyme used to digest the zona pellucida is a protease, a pronase or a combination thereof. More preferably, the enzyme is a pronase.

Preferably, the pronase is used at a concentration between 0.1 to 5%. More preferably, the concentration is between 0.25% to 2%. Most preferably, the pronase is at a concentration of about 0.5%.

It will be appreciated by those skilled in the art that any procedure of enucleation of the oocyte can be performed, including, aspiration, physical removal, use of DNA-specific fluorochromes, and irradiation with ultraviolet light. Preferably, the enucleation is by physical means. Most preferable, the physical means is bisection.

Preferably, the step of transferring the MCT or MCT nuclei is by fusion. More preferably, the method of fusion is selected from the group consisting of chemical fusion, electrofusion and biofusion. Preferably, the chemical fusion or biofusion is accomplished by exposing the enucleated oocyte and MCT combination to a fusion agent. Preferably, the fusion agent is any compound or biological organism that can increase the probability that portions of plasma membranes from different cells will fuse when an MCT donor is placed adjacent to the enucleated oocyte recipient. Most preferably, the fusion agents are selected from the group consisting of polyethylene glycol (PEG), trypsin, dimethylsulfoxide (DMSO), lectins, agglutinin, viruses, and Sendai virus.

The electrofusion is preferably induced by application of an electrical pulse across the contact/fusion plane. More preferably, the electrofusion comprises the step of delivering one or more electrical pulses to the enucleated oocyte and MCT combination.

Also provided by the present invention are mammals obtained according to the above methods, and offspring of those mammals.

### BRIEF DESCRIPTION OF THE FIGURES

FIG. 1 shows the selective growth stimulation of MCTs by high density/high serum culture. Standard cell culture techniques leads to a successive loss of the MCT population and result in a conventional fibroblast culture.

FIG. 2 shows the activation of Oct4-promoter in somatic explants of Oct4-eGFP tg mice. Genital ridge of a male fetus (day 14.5 p.c.) with massive expression of GFP in the primordial germ cells is shown under fluorescent (A) and brightfield optics (B). Bar=150 μm. Outgrowth of mesenchymal explant, under fluorescent (C) and brightfield (D) optics after 2 days of culture. No GFP positive cells were found. In the upper left the explant is visible. After 8 days in culture several GFP positive cells were detectable within the outgrowth (E, F), bar=140 μm. Confocal analysis of murine MCTs cultured in high serum, G) fluorescent, H) brightfield and I) merged images, bar=10 μm. The GFP is preferentially located in the cytoplasm, probably because it does not contain a nuclear localisation motif. J) shows RT-PCR detection of native Oct4 transcripts in MCTS; M, DNA ladder; lane 1, MCTS; lane 2, no-RT control of 1; lane 3, ES cells; lane 4, no-RT control of 3; lane 5, no template control.

FIG. 3 shows the induction of 3D-growth and AP positive cells in porcine MCTS. A and B show the high serum (30%) induction of 3D-colony growth (passage 3, 5d) in porcine fetal fibroblasts. C shows the control culture of the same cell batch cultured in standard medium (10% FCS, 5 d). D shows BrdU incorporation in high serum cultures (5 d, 30% FCS). Note that only cells within the 3D-colonies (arrows) incorporated BrdU, the surrounding monolayer is unlabelled, inset: another 3D-colony. E shows BrdU incorporation in proliferating fibroblasts (3 d, standard medium with 10% FCS), the majority of the cells is labelled. F shows BrdU incorporation in confluent fibroblasts (5 d, standard medium), the majority of the cells became contact-inhibited and stopped to proliferate. G-J shows the induction of AP-positive cells, accompanied with 3D-colony growth after 2, 4, 6, 8 days in high serum culture. K shows the higher magnification of AP positive cells aggregated in 3D-colony (4 d). L shows individual AP-positive cells within the fibroblast monolayer. Bars=20 μm.

FIG. 4 shows the induction of AP-positive 3D-colonies in fetal and adult fibroblast cultures. A shows porcine fibroblasts from fetal and adult origin of the same batches, respectively, were split and cultured with high serum (30%) or standard (10% FCS) conditions in 6-well plates, after 5 days the cultures were fixed and stained for endogenous AP activity. Note the massive induction of AP-positive 3D-colonies in the fetal culture (red dots). B shows the induction of 3D-colony growth and AP is reversible. After six passages with constant 3D-colony formation and AP expression in high serum (30% FCS) fetal cells were trypsinised, replated and cultured for two passages with standard medium (10% FCS) before AP-staining.

FIG. 5 shows the proliferative induction by high serum culture. A shows the growth curves of fetal fibroblasts cultured in standard medium (?) containing 10% FCS and high serum medium (?) containing 30% FCS. Cells were enumerated at each passage under a hemocytometer. B shows the mean cell number per passage (±SD) of fibroblasts from the same batch cultured in DMEM with 10% (?) or 30% (?) FCS after 6 days passage. C shows the cell cycle status in standard and high serum culture. Note that the high serum culture displays a normal ploidy.

FIG. 6 shows the anchorage-independent growth of MCTs in suspension culture. High serum induced 3D-colonies were isolated, trypsinised to single cell suspensions and seeded into bacteriological dishes to prevent attachment. A shows that tiny aggregates formed in HS culture medium without supplementation. B shows that HS medium supplemented with retinoic acid (10⁻⁷ M) the initial aggregates reattach and show outgrowing cells on the surface. C shows that spheroids of >300 μm grow over 10-15 days in HS medium supplemented with dexamethasone (10⁻⁷M), inset: lower magnification. D shows that dexamethasone-spheroids stained for endogenous AP, bar=230 μm. E shows that expression of vimentin in fibroblasts cultured in standard medium (passage 5), merged image of antibody (red) and nuclei (blue) staining. Loss of vimentin reactivity in cells derived from dexamethasone-spheroids. After 15 days of suspension culture the spheroids were allowed to reattach to gelatinised coverslips and probed with a monoclonal anti-vimentin antibody.

FIG. 7 shows a whole mount staining for LacZ activity in a control fetus (left) and a fetus (d15.5 p.c.) derived from a MCTs (Rosa26/OG2) injected blastocyst (right). Note the β-galactosidase staining in liver (arrow) and genital ridge (arrowheads) of the chimeric fetus.

FIG. 8 shows Oct-4 promoter driven expression of GFP in the genital ridges of a chimeric fetus (d15.5 p.c.) derived from a MCTs (Rosa26/OG2) injected blastocyst (left and middle). Genital ridge from a control OG2/Rosa26 fetus (right).

### DETAILED DESCRIPTION OF THE INVENTION

Before describing the present invention in detail, it is to be understood that this invention is not limited to particularly exemplified cell culture techniques, serum, media or methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

It must be noted that as used herein and in the appended claims, the singular forms “a,” “an,” and “the” include plural reference unless the context clearly dictates otherwise. Thus, for example, a reference to “a cell” includes a plurality of such cells, and a reference to “an oocyte” is a reference to one or more oocytes, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention relates to methods of producing primary cell cultures. The term “primary cell culture” denotes a mixed cell population of cells that permits interaction of many different cell types isolated from a tissue. The word “primary” takes its usual meaning in the art of tissue culture. For example, a primary culture of epidermal tissue may allow the interaction between mesenchymal and epithelial cells.

The primary cell culture is produced from tissue biopsy material. The term “tissue” refers to a group or layer of similarly specialised cells, which together perform certain special functions. Accordingly, the term “tissue biopsy” as used herein refers to a specimen obtained by removing a group or layer of similarly specialised cells from animals for use in primary cell culture. The term includes aspiration biopsies; brush biopsies; chorionic villus biopsies; endoscopic biopsies; excision biopsies; needle biopsies (specimens obtained by removal by aspiration through an appropriate needle or trocar that pierces the skin, or the external surface of an organ, and into the underlying tissue to be examined); open biopsies; punch biopsies (trephine); shave biopsies; sponge biopsies; and wedge biopsies.

The tissue biopsy may be taken from any animal, for which the study of tissue-specific progenitor cells or stem cell-like cells is required. Suitable mammalian animals include members of the Orders Primates, Rodentia, Lagomorpha, Cetacea, Carnivora, Perissodactyla and Artiodactyla. Members of the Orders Perissodactyla and Artiodactyla are particularly preferred because of their similar biology and economic importance.

For example, Artiodactyla comprise approximately 150 living species distributed through nine families: pigs (Suidae), peccaries (Tayassuidae), hippopotamuses (Hippopotamidae), camels (Camelidae), chevrotains (Tragulidae), giraffes and okapi (Giraffidae), deer (Cervidae), pronghorri (Antilocapridae), and cattle, sheep, goats and antelope (Bovidae). Many of these animals are used as feed animals in various countries. More importantly, with respect to the present invention, many of the economically important animals such as goats, sheep, cattle and pigs have very similar biology and share high degrees of genomic homology.

The Order Perissodactyla comprises horses and donkeys, which are both economically important and closely related. Indeed, it is well known that horses and donkeys interbreed.

In one embodiment, the tissue biopsies will be obtained from ungulates, and in particular, bovids, ovids, cervids, suids, equids and camelids. Examples of such representatives are cows or bulls, bison, buffalo, sheep, big-horn sheep, horses, ponies, donkeys, mule, deer, elk, caribou, goat, water buffalo, camels, llama, alpaca, and pigs. Especially preferred bovine species are Bos taurus, Bos indicus, and Bos buffaloes cows or bulls.

In another embodiment, the tissue biopsies will be obtained from primates, especially humans.

The general purpose of the primary cell culture is to “isolate,” “proliferate” or “selectively expand” tissue-specific progenitor cells or stem cell-like cells present in a tissue biopsy. The terms “isolate,” “proliferate” or “selectively expand” as used herein refers to the culturing process by which the tissue-specific progenitor cells or stem cell-like cells are increased in number relative to the other cells present in the tissue biopsy.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to mesenchymal connective tissue derived stem cell-like cells (MCTs). The characteristics of MCTs are reminiscent of pluripotent stem cells. The MCTs are characterised by loss of contact inhibition, anchorage independent growth, de novo expression of alkaline phosphatase and activation of the germ line specific Oct4 promoter. The proliferative potential of these cells is significantly increased compared to primary fibroblasts.

In one embodiment the MCT is the MCT deposited under the Budapest Treaty at the Deutsche Sammlung Von Mikroorganismen und Zellkulturen GmbH (DSMZ), Germany on September 2004, under accession number #12345.

After the tissue biopsy has been obtained, the initial step in the isolation, proliferation or selective expansion of the tissue-specific progenitor cells, stem cell-like cell or MCT present in a tissue biopsy involves the culturing of the tissue biopsy. The terms “culture,” “cultured” and “culturing” are used herein interchangeably, to refer to the process by which the tissue biopsy is grown in vitro.

The tissue biopsy is preferably subjected to physical and/or chemical dissociating means capable of dissociating cellular stratum in the tissue sample. Methods for dissociating cellular layers within the tissues are well known in the field. For example, the dissociating means may be either a physical or a chemical disruption means. Physical dissociation means might include, for example, scraping the tissue biopsy with a scalpel, mincing the tissue, physically cutting the layers apart, or perfusing the tissue with enzymes. Chemical dissociation means might include, for example, digestion with enzymes such as trypsin, dispase, collagenase, trypsin-EDTA, thermolysin, pronase, hyaluronidase, elastase, papain and pancreatin. Non-enzymatic solutions for the dissociation of tissue can also be used.

In one embodiment, dissociation of the tissue biopsy is achieved by placing the tissue biopsy in a pre-warmed enzyme solution containing an amount of trypsin sufficient to dissociate the cellular stratum in the tissue biopsy. Preferably, the enzyme solution used in the method is calcium and magnesium free.

Where the tissue biopsy is derived from an animals skin (comprising epithelial and dermal cells) the amount of trypsin that might be used in the method is preferably between about 5 and 0.1% trypsin per volume of solution. Desirable the trypsin concentration of the solution is about 2.5 to 0.25%, with about 0.5% trypsin being most preferred.

The time period over which the tissue biopsy is subjected to the trypsin solution may vary depending on the size of the tissue biopsy taken. Preferably the tissue biopsy is placed in the presence of the trypsin solution for sufficient time to weaken the cohesive bonding between the tissue stratum. For example, where the tissue sample is taken from an animal's skin the tissue biopsy might be placed in trypsin for between 5 to 60 minutes. In one embodiment, the tissue biopsy is immersed in the trypsin solution for between 10 and 30 minutes with 15 to 20 minutes being optimal for most tissue biopsies.

After the tissue biopsy has been immersed in the trypsin solution for an appropriate amount of time, the dissociated cells are removed and suspended in tissue culture medium. The terms “culture media,” “tissue culture media” or “tissue culture medium” are recognised in the art, and refers generally to any substance or preparation used for the cultivation of living cells. There are a large number of tissue culture media that exist for culturing tissue from animals. Some of these are complex and some are simple. Examples of media that would be useful in the present invention include Modified Eagle's Medium (MEM), Dulbecco's Modified Eagle's Medium (DMEM), RPMI 1640, F-12, IMDM, Alpha Medium and McCoy's Medium. Most preferably, the culture medium is DMEM.

In one embodiment, enzymatically dissociated and eviscerated fetuses or mesenchymal explant (<1 mm³) cultures of connective tissue are suspended in DMEM supplemented with 1 mM glutamine, 1% non-essential amino acids, 1% vitamin solution, 0.1 mM mercaptoethanol, 100 U/ml penicillin, and 100 mg/ml streptomycin (all from Sigma, Deisenhofen, Germany).

In order to encourage the tissue-specific progenitor cells or stem cell-like cells to proliferate, serum is added to the tissue culture medium. The serum in the culture medium may be allogeneic serum (ie., from the same animal species, but not the same animal), autologous serum (ie., from the same animal) or xenogeneic serum (ie., from a different animal species). In one embodiment, heat-inactivated autologous serum is used.

When the dissociated tissue biopsy is initially cultured the amount of serum used is typically about 10%. The term “about” as used herein to describe the amount of serum used in the culture medium indicates that in certain circumstances the amount of serum used will be slightly more (approximately 10% more) or slightly less (approximately 10% less), than the stated amount. For example, about 10% serum would mean that as little as 9% serum might be used or up to a maximum of 11% serum. About 30% serum would mean that as little as 27% serum might be used serum (i.e. within 10% of the stated volume) or as much as 33% serum (i.e. within 10% of the stated volume).

The dissociated tissue biopsy cells, including the tissue-specific progenitor cells or stem cell-like cells are incubated in a humidified 95% air/5% C0₂ atmosphere at 37° C.

After the second passage of the cells after setting up the culture, the serum concentration is adjusted to about 30%. The precise timing of this stage is difficult to predict as this will vary depending upon the type of tissue used and the age of the material. For example, fetal tissue is typically faster growing than adult tissue. The presence of the increased serum concentration enables the tissue-specific progenitor cells or stem cell-like cells to proliferate, while the other cells present such as keratinocytes, basal cells, Langerhans cells, fibroblasts and melanocytes, have depressed growth. Approximately, every 48 hours or so, 50% of the culture medium is preferably replaced with fresh medium.

As the tissue-specific progenitor cells or stem cell-like cells proliferate they generally take on a 3D appearance. Once the 3D-colonies reach approximately 200-300 μm in diameter they are isolated and trypsinised to obtain single cells suspensions. Subsequently, 10⁴ cells are seeded into bacteriological culture dishes to prevent attachment. Supplementation of the culture medium (DMEM/30% FCS) with dexamethasone results in aggregations of small multicellular spheroids usually within 24 hours, which continue to grow up to a diameter of >400 μm after 10-15 days.

The maximal replicative limit can be determined by serially subpassaging the cells as 12.5×10³ cell aliquots seeded per cm² in 6-well-dishes, trysinised after 5-7 days, counted and reseeded.

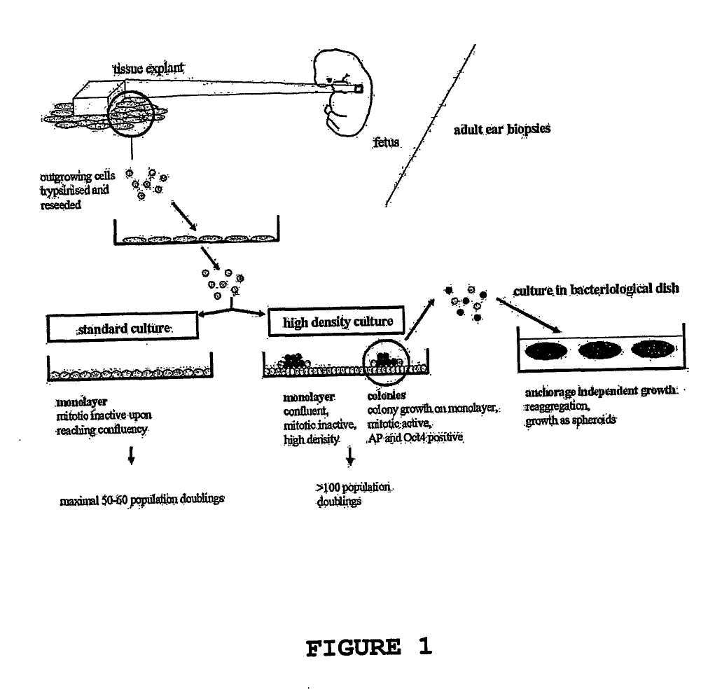

In one embodiment, the tissue-specific progenitor cells or stem cell-like cells are mesenchymal connective tissue derived stem cell-like cells (MCTs). The MCTs show several characteristics not found in fibroblasts, e.g. they have a significantly extended proliferative capacity of >100 cell doublings in vitro. This allows an extended amplification of clonal cell strains or mass cultures and could simplify genetic modifications and potentially enables two rounds of genetic modifications and selection. Also enough cells for grafting procedures can be obtained, as MCTs might be suitable for directed differentiation into several cell types. FIG. 1 shows the selective growth stimulation of MCTs by high density/high serum culture. Standard cell culture techniques leads to a successive loss of the MCT population and result in a conventional fibroblast culture. One specific type of MCT has been deposited under the Budapest Treaty at the Deutsche Sammlung Von Mikroorganismen und Zellkulturen GmbH (DSMZ), Germany on September 2004, under accession number #12345.

Once the tissue-specific progenitor cells, stem cell-like cells or MCTs have been isolated or proliferated they can then be used, for example, for direct transplantation or to produce differentiated cells in vitro for transplantation or in nuclear transfer techniques. The invention accordingly provides, for example, stem cells that may serve as a source for many other, more differentiated cell types.

One embodiment pertains to the progeny of the tissue-specific progenitor cells, stem cell-like cells or MCTs, e.g. those cells which have been derived from the cells of the initial tissue biopsy. Such progeny can include subsequent generations of tissue-specific progenitor cells, stem cell-like cells or MCTs, as well as lineage committed cells generated by inducing differentiation of the tissue-specific progenitor cells, stem cell-like cells or MCTs after their isolation from the tissue biopsy, e.g., induced in vitro.

Another embodiment relates to cellular compositions enriched for tissue-specific progenitor cells, stem cell-like cells or MCTs, or the progeny thereof. In certain embodiments, the cells will be provided as part of a pharmaceutical preparation, e.g., a sterile, free of the presence of unwanted virus, bacteria and other pathogens, as well as pyrogen-free preparation. That is, for animal administration, the tissue-specific progenitor cells, stem cell-like cells or MCTs should meet sterility, pyrogenicity, general safety and purity standards as required by FDA Office of Biologics standards.

In certain embodiments, such cellular compositions can be used for transplantation into animals, preferably mammals, and even more preferably humans. The tissue-specific progenitor cells, stem cell-like cells or MCTs can be autologous, allogeneic or xenogeneic with respect to the transplantation host.

Yet another aspect of the present invention concerns cellular compositions, which include as a cellular component, substantially pure preparations of the tissue-specific progenitor cells, stem cell-like cells or MCTs, or the progeny thereof. Cellular compositions of the present invention include not only substantially pure populations of the tissue-specific progenitor cells, stem cell-like cells or MCTs, but can also include cell culture components, e.g., culture media including amino acids, metals, coenzyme factors, as well as small populations of non-tissue-specific progenitor cells, stem cell-like cells or MCTs cells, e.g., some of which may arise by subsequent differentiation of isolated tissue-specific progenitor cells, stem cell-like cells or MCTs of the invention. Furthermore, other non-cellular components include those which render the cellular component suitable for support under particular circumstances, eg., implantation, eg., continuous culture.

As common methods of administering the tissue-specific progenitor cells, stem cell-like cells or MCTs of the present invention to animals, particularly humans, which are described in detail herein, include injection or implantation of the tissue-specific progenitor cells, stem cell-like cells or MCTs into target sites in the animals, the cells of the invention can be inserted into a delivery device which facilitates introduction by, injection or implantation, of the cells into the animals. Such delivery devices include tubes, eg., catheters, for injecting cells and fluids into the body of a recipient animal. In a preferred embodiment, the tubes additionally have a needle, eg., a syringe, through which the cells of the invention can be introduced into the animal at a desired location. The tissue-specific progenitor cells, stem cell-like cells or MCTs of the invention can be inserted into such a delivery device, eg., a syringe, in different forms. For example, the cells can be suspended in a solution or embedded in a support matrix when contained in such a delivery device. As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating tissue-specific progenitor cells, stem cell-like cells or MCTs as described herein in a pharmaceutically acceptable carrier or diluent and, as required, other ingredients enumerated above, followed by filtered sterilisation.

Support matrices in which the tissue-specific progenitor cells, stem cell-like cells or MCTS can be incorporated or embedded include matrices which are recipient-compatible and which degrade into products which are not harmful to the recipient. Natural and/or synthetic biodegradable matrices are examples of such matrices. Natural biodegradable matrices include plasma clots, eg., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. See eg., U.S. Pat. Nos. 4,298,002 and 5,308,701. These matrices provide support and protection for the fragile progenitor cells in vivo and are, therefore, the preferred form in which the tissue-specific progenitor cells, stem cell-like cells or MCTs are introduced into the recipient animals.

The present invention also provides substantially pure tissue-specific progenitor cells, stem cell-like cells or MCTs cells which can be used therapeutically for treatment of various disorders.

To illustrate, the tissue-specific progenitor cells, stem cell-like cells or MCTs of the invention can be used in the treatment or prophylaxis of a variety of disorders. For instance, the tissue-specific progenitor cells, stem cell-like cells or MCTs can be used to produce populations of differentiated cells for repair of damaged tissue eg pancreatic tissue, cardiac tissue, nerves and the like. Likewise, such cell populations can be used to regenerate or replace pancreatic tissue, cardiac tissue or nerves lost due to, pancreatolysis, eg., destruction of pancreatic tissue, such as pancreatitis, heart disease or neuropathy.

Yet another embodiment provides methods for screening various compounds for their ability to modulate growth, proliferation or differentiation of tissue-specific progenitor cells, stem cell-like cells or MCTs. In an illustrative embodiment, the subject tissue-specific progenitor cells, stem cell-like cells or MCTs, and their progeny, can be used to screen various compounds or natural products. Such explants can be maintained in minimal culture media for extended periods of time (eg., for 7-21 days or longer) and can be contacted with any compound, eg., small molecule or natural product, eg., growth factor, to determine the effect of such compound on one of cellular growth, proliferation or differentiation of the tissue-specific progenitor cells, stem cell-like cells or MCTs. Detection and quantification of growth, proliferation or differentiation of these cells in response to a given compound provides a means for determining the compound's efficacy at inducing one of the growth, proliferation or differentiation. Methods of measuring cell proliferation are well known in the art and most commonly include determining DNA synthesis characteristic of cell replication. There are numerous methods in the art for measuring DNA synthesis, any of which may be used according to the invention. In an embodiment of the invention, DNA synthesis has been determined using a radioactive label (³H-thymidine) or labelled nucleotide analogues (BrdU) for detection by immunofluorescence. The efficacy of the compound can be assessed by generating dose response curves from data obtained using various concentrations of the compound. A control assay can also be performed to provide a baseline for comparison. Identification of the progenitor cell population(s) amplified in response to a given test agent can be carried out according to such phenotyping as described above.

In one embodiment, the tissue-specific progenitor cells, stem cell-like cells or MCTs are used for cloning mammals by nuclear transfer or nuclear transplantation. In the subject application, the terms “nuclear transfer” or “nuclear transplantation” are used interchangeably; however, these terms as used herein refers to introducing a full complement of nuclear DNA from one cell to an enucleated cell.

The first step in the preferred methods involves the isolation of a recipient oocyte from a suitable animal. In this regard, the oocyte may be obtained from any animal source and at any stage of maturation. Methods for isolation of oocytes are well known in the art. For example, oocytes can be isolated from either oviducts and/or ovaries of live animals by oviductal recovery procedures or transvaginal oocyte recovery procedures well known in the art. See, eg., Pieterse et al., 1988, “Aspiration of bovine oocytes during transvaginal ultrasound scanning of the ovaries,” Theriogenology 30: 751-762. Furthermore, oocytes can be isolated from ovaries or oviducts of deceased animals. For example, ovaries can be obtained from abattoirs and the oocytes aspirated from these ovaries. The oocytes can also be isolated from the ovaries of a recently sacrificed animal or when the ovary has been frozen and/or thawed.

Briefly, in one preferred embodiment, immature (prophase I) oocytes from mammalian ovaries are harvested by aspiration. For the successful use of techniques such as genetic engineering, nuclear transfer and cloning, once these oocytes have been harvested they must generally be matured in vitro before these cells may be used as recipient cells for nuclear transfer.

The stage of maturation of the oocyte at enucleation and nuclear transfer has been reported to be significant to the success of nuclear transfer methods. (See eg., Prather et al., Differentiation, 48, 1-8, 1991). In general, successful mammalian embryo cloning practices use the metaphase II stage oocyte as the recipient oocyte because at this stage it is believed that the oocyte can be or is sufficiently activated to treat the introduced nucleus as it does a fertilising sperm.

The in vitro maturation of oocytes usually takes place in a maturation medium until the oocyte has extruded the first polar body, or until the oocyte has attained the metaphase II stage. In domestic animals, and especially cattle, the oocyte maturation period generally ranges from about 16-52 hours, preferably about 28-42 hours and more preferably about 18-24 hours post-aspiration. For purposes of the present invention, this period of time is known as the “maturation period.”

Oocytes can be matured in a variety ways and using a variety of media well known to a person of ordinary skill in the art. See, eg., U.S. Pat. No. 5,057,420; Saito et al., 1992, Roux's Arch. Dev. Biol. 201: 134-141 for bovine organisms and Wells et al., 1997, Biol. Repr. 57: 385-393 for ovine organisms and WO97/07668, entitled “Unactivated Oocytes as Cytoplast Recipients for Nuclear Transfer,” all hereby incorporated herein by reference in the entirety, including all figures, tables, and drawings.

One of the most common media used for the collection and maturation of oocytes is TCM-199, and 1 to 20% serum supplement including fetal calf serum (FCS), newborn serum, estrual cow serum, lamb serum or steer serum. Example 1 shows one example of a preferred maintenance medium: TCM-199 with Earl salts supplemented with 15% cow serum and including 10IU/ml pregnant mare serum gonadotropin and 5IU/ml human chorionic gonadotropin (Suigon^{R} Vet, Intervet, Australia). Oocytes can be successfully matured in this type of medium within an environment comprising 5% CO₂ at 39° C.

While it will be appreciated by those skilled in the art that freshly isolated and matured oocytes are preferred, it will also be appreciated that it is possible to cryopreserve the oocytes after harvesting or after maturation. Accordingly, the term “cryopreserving” as used herein can refer to freezing an oocyte, a cell, embryo, or animal of the invention. The oocytes, cells, embryos, or portions of animals of the invention are frozen at temperatures preferably lower than 0° C., more preferably lower than −80° C., and most preferably at temperatures lower than −196° C. Oocytes, cells and embryos in the invention can be cryopreserved for an indefinite amount of time. It is known that biological materials can be cryopreserved for more than fifty years. For example, semen that is cryopreserved for more than fifty years can be utilised to artificially inseminate a female bovine animal. Methods and tools for cryopreservation are well known to those skilled in the art. See, eg., U.S. Pat. No. 5,160,312, entitled “Cryopreservation Process for Direct Transfer of Embryos”.

If cyropreserved oocytes are utilised then these must be initially thawed before placing the oocytes in maturation medium. Methods of thawing cryopreserved materials such that they are active after the thawing process are well-known to those of ordinary skill in the art.

In a further preferred embodiment, mature (metaphase II) oocytes, which have been matured in vivo, are harvested and used in the nuclear transfer methods disclosed herein. Essentially, mature metaphase II oocytes are collected surgically from either non-superovulated or superovulated cows or heifers 35 to 48 hours past the onset of estrus or past the injection of human chorionic gonadotropin (hCG) or similar hormone.

Where oocytes have been cultured in vitro cumulus cells that may have accumulated may be removed to provide oocytes that are at a more suitable stage of maturation for enucleation. Cumulus cells may be removed by pipetting or vortexing, for example, in the presence of 0.5% hyaluronidase.

After the maturation period as described above the zona pellucida may be removed from the oocytes if desired. The advantages of zona pellucida removal are described in PCT/AU02/00491, which is incorporated in its entirety herein by reference. The removal of the zona pellucida from the oocyte may be carried out by any method known in the art including physical manipulation (mechanical opening), chemical treatment or enzymatic digestion (Wells and Powell, 2000). Physical manipulation may involve the use of a micropipette or a microsurgical blade. Preferably, enzymatic digestion is used.

In one particularly preferred embodiment, the zona pellucida is removed by enzymatic digestion in the presence of a protease or pronase. Briefly, mature oocytes are placed into a solution comprising a protease, pronase or combination of each at a total concentration in the range of 0.1% - 5%, more preferably 0.25% -2% and most preferably about 0.5%. The mature oocyte is then allowed to incubate at between 30° C. to about 45° C., preferably about 39° C. for a period of 1 to 30 minutes. Preferably the oocytes are exposed to the enzyme for about 5 minutes. Although pronase may be harmful to the membranes of oocytes, this effect may be minimised by addition of serum such as FCS or cow serum. The unique advantage of zona removal with pronase is that no individual treatment is required, and the procedure can be performed in quantities of 100's of oocytes. Once the zona pellucida has been removed the zona pellucida-free mature oocyte are rinsed in 4 ml Hepes buffered TCM-199 medium supplemented with 20% FCS and 10 μg/ml cytochalasin B and then enucleated.

The terms “enucleation”, “enucleated” and “enucleated oocyte” are used interchangeably herein and refers to an oocyte which has had part of its contents removed.

Enucleation of the oocyte may be achieved physically, by actual removal of the nucleus, pronuclei or metaphase plate (depending on the oocyte), or functionally, such as by the application of ultraviolet radiation or another enucleating influence. All of these methods are well known to those of ordinary skill in the art. For example, physical means includes aspiration (Smith & Wilmut, Biol. Reprod., 40: 1027-1035 (1989)); functional means include use of DNA-specific fluorochromes (See, for example, Tsunoda et al., J. Reprod. Fertil. 82: 173 (1988)), and irradiation with ultraviolet light (See, for example, Gurdon, Q. J. Microsc. Soc., 101: 299-311 (1960)). Enucleation may also be effected by other methods known in the art. See, for example, U.S. Pat. No. 4,994,384; U.S. Pat. No. 5,057,420; and Willadsen, 1986, Nature 320:63-65, herein incorporated by reference.

Preferably, the oocyte is enucleated by means of manual bisection. Oocyte bisection may be carried out by any method known to those skilled in the art. In one preferred embodiment, the bisection is carried out using a microsurgical blade as described in International Patent Application No. WO98/29532 which is incorporated by reference herein. Briefly, oocytes are split asymmetrically into fragments representing approximately 30% and 70% of the total oocyte volume using an ultra sharp splitting blade (AB Technology, Pullman, W A, USA). The oocytes may then be screened to identify those of which have been successfully enucleated. This screening may be effected by staining the oocytes with 1 microgram per millilitre of the Hoechst fluorochrome 33342 dissolved in TCM-199 media supplemented with 20% FCS, and then viewing the oocytes under ultraviolet irradiation with an inverted microscope for less than 10 seconds. The oocytes that have been successfully enucleated (demi-oocytes) can then be placed in a suitable culture medium, eg., TCM-199 media supplemented with 20% FCS.

In the present invention, the recipient oocytes will preferably be enucleated at a time ranging from about 10 hours to about 40 hours after the initiation of in vitro maturation, more preferably from about 16 hours to about 24 hours after initiation of in vitro maturation, and most preferably about 16-18 hours after initiation of in vitro maturation.

The bisection technique described herein requires much less time and skill than other methods of enucleation and the subsequent selection by staining results in high accuracy. Consequently, for large-scale application of cloning technology the present bisection technique can be more efficient than other techniques.

A single tissue-specific progenitor cell, stem cell-like cell or MCTs of the present invention of the same species as the enucleated oocyte can then be transferred by fusion into the enucleated oocyte thereby producing a reconstituted cell.

Analysis of cell cycle stage may be performed as described in Kubota et al., PNAS 97: 990-995 (2000). Briefly, cell cultures at different passages are grown to confluency. After trypsinisation, cells are washed with TCM-199 plus 10% FCS and re-suspended to a concentration of 5×10⁵ cells/ml in 1 ml PBS with glucose (6.1 mM) at 4° C. Cells are fixed overnight by adding 3 ml of ice-cold ethanol. For nuclear staining, cells are then pelleted, washed with PBS and re-suspended in PBS containing 30 μg/ml propidium iodide and 0.3 mg/ml RNase A. Cells are allowed to incubate for 1 h at room temperature in the dark before filtered through a 30 μm mesh. Cells are then analyzed.

To examine the ploidy of the tissue-specific progenitor cells, stem cell-like cells or MCTs at various passages, chromosome counts may be determined at different passages of culture using standard preparation of metaphase spreads (See, for example, Kubota et al., PNAS 97: 990-995 (2000)).

Cultured tissue-specific progenitor cells, stem cell-like cells or MCTs may also be genetically altered by transgenic methods well-known to those of ordinary skill in the art. See, for example, Molecular cloning a Laboratory Manual, 2nd Ed., 1989, Sambrook, Fritsch and Maniatis, Cold Spring Harbor Laboratory Press; U.S. Pat. No. 5,612,205; U.S. Pat. No. 5,633,067; EPO 264 166, entitled “Transgenic Animals Secreting Desired Proteins Into Milk”; WO94/19935, entitled “Isolation of Components of Interest From Milk”; WO93/22432, entitled “Method for Identifying Transgenic Pre-implantation Embryos”; and WO95/175085, entitled “Transgenic Production of Antibodies in Milk,” all of which are incorporated by reference herein in their entirety including all figures, drawings and tables. Any known method for inserting, deleting or modifying a desired gene from a mammalian cell may be used for altering the tissue-specific progenitor cells, stem cell-like cells or MCTs to be used as the nuclear donor. These procedures may remove all or part of a gene, and the gene may be heterologous. Included is the technique of homologous recombination, which allows the insertion, deletion or modification of a gene or genes at a specific site or sites in the cell genome.

Examples for modifying a target DNA genome by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

The present invention can thus be used to provide adult mammals with desired genotypes. Multiplication of adult ungulates with proven genetic superiority or other desirable traits is particularly useful, including transgenic or genetically engineered animals, and chimeric animals. Furthermore, cell and tissues from the nuclear transfer fetus, including transgenic and/or chimeric fetuses, can be used in cell, tissue and organ transplantation.

Methods for generating transgenic cells typically include the steps of (1) assembling a suitable DNA construct useful for inserting a specific DNA sequence into the nuclear genome of tissue-specific progenitor cells, stem cell-like cells or MCTs; (2) transfecting the DNA construct into the tissue-specific progenitor cells, stem cell-like cells or MCTs; (3) allowing random insertion and/or homologous recombination to occur. The modification resulting from this process may be the insertion of a suitable DNA construct(s) into the target genome; deletion of DNA from the target genome; and/or mutation of the target genome.

DNA constructs can comprise a gene of interest as well as a variety of elements including regulatory promoters, insulators, enhancers, and repressors as well as elements for ribosomal binding to the RNA transcribed from the DNA construct.

DNA constructs can also encode ribozymes and anti-sense DNA and/or PNA, identified previously herein. These examples are well known to a person of ordinary skill in the art and are not meant to be limiting.

Due to the effective recombinant DNA techniques available in conjunction with DNA sequences for regulatory elements and genes readily available in data bases and the commercial sector, a person of ordinary skill in the art can readily generate a DNA construct appropriate for establishing transgenic cells using the materials and methods described herein.

Transfection techniques are well known to a person of ordinary skill in the art and materials and methods for carrying out transfection of DNA constructs into cells are commercially available. Materials typically used to transfect cells with DNA constructs are lipophilic compounds, such as Lipofectin™ for example. Particular lipophilic compounds can be induced to form liposomes for mediating transfection of the DNA construct into the cells.

Target sequences from the DNA construct can be inserted into specific regions of the nuclear genome by rational design of the DNA construct. These design techniques and methods are well known to a person of ordinary skill in the art. See, for example, U.S. Pat. No. 5,633,067; U.S. Pat. No. 5,612,205 and PCT publication WO93/22432, all of which are incorporated by reference herein in their entirety. Once the desired DNA sequence is inserted into the nuclear genome, the location of the insertion region as well as the frequency with which the desired DNA sequence has inserted into the nuclear genome can be identified by methods well known to those skilled in the art.

Once the transgene is inserted into the nuclear genome of the donor tissue-specific progenitor cells, stem cell-like cells or MCTs, that cell, like other donor tissue-specific progenitor cells, stem cell-like cells or MCTs of the invention, can be used as a nuclear donor in nuclear transfer methods. The means of transferring the nucleus of a tissue-specific progenitor cells, stem cell-like cells or MCTs into the enucleated oocyte preferably involves cell fusion to form a reconstituted cell.

Fusion is typically induced by application of a DC electrical pulse across the contact/fusion plane, but additional AC current may be used to assist alignment of donor and recipient cells. Electrofusion produces a pulse of electricity that is sufficient to cause a transient breakdown of the plasma membrane and which is short enough that the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including eg., sucrose, mannitol, sorbitol and phosphate buffered solution.

Fusion can also be accomplished using Sendai virus as a fusogenic agent (Graham, Wister Inot. Symp. Monogr., 9, 19, 1969). Fusion may also be induced by exposure of the cells to fusion-promoting chemicals, such as polyethylene glycol.

Preferably, the donor tissue-specific progenitor cells, stem cell-like cells or MCTs and enucleated oocyte are placed in a 500 μm fusion chamber and covered with 4 ml of 26° C.-27° C. fusion medium (0.3M mannitol, 0.1 mM MgSO₄, 0.05 mM CaCl₂). The cells are then electrofused by application of a double direct current (DC) electrical pulse of 70-100V for about 15 μs, approximately 1 s apart. After fusion, the resultant fused reconstituted cells are then placed in a suitable medium until activation, eg., TCM-199 medium.

In a preferred method of cell fusion the donor tissue-specific progenitor cell, stem cell-like cell or MCT is firstly attached to the enucleated oocyte. For example, a compound is selected to attach the progenitor cell, stem cell-like cell or MCT to the enucleated oocyte to enable fusing of the donor cell and enucleated oocyte membranes. The compound may be any compound capable of agglutinating cells. The compound may be a protein or glycoprotein capable of binding or agglutinating carbohydrate. More preferably the compound is a lectin. The lectin may be selected from the group including Concanavalin A, Canavalin A, Ricin, soybean lectin, lotus seed lectin and phytohemaglutinin (PHA). Preferably the compound is PHA.

In one preferred embodiment, the method of electrofusion described above also comprises a further fusion step, or the fusion step comprises described above comprises one donor progenitor cell, stem cell-like cell or MCT and two or more enucleated oocytes. The double fusion method has the advantageous effect of increasing the cytoplasmic volume of the reconstituted cell.

A reconstituted cell is typically activated by electrical and/or non-electrical means before, during, and/or after fusion of the nuclear donor and recipient oocyte (See, for example, Susko-Parrish et al., U.S. Pat. No. 5,496,720). Activation methods include:
1). Electric pulses;
2). Chemically induced shock;
3). Penetration by sperm;
4). Increasing levels of divalent cations in
   the oocyte by introducing divalent cations into the oocyte cytoplasm, eg., magnesium, strontium, barium or calcium, eg., in the form of an ionophore. Other methods of increasing divalent cation levels include the use of electric shock, treatment with ethanol and treatment with caged chelators; and
5). Reducing phosphorylation of cellular proteins in the oocyte by known methods, eg., by the addition of kinase inhibitors, eg., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, eg., phosphatase 2A and phosphatase 2B.

The activated reconstituted cells, or embryos, are typically cultured in medium well known to those of ordinary skill in the art, and include, without limitation, TCM-199 plus 10% FSC, Tyrodes-Albumin-Lactate-Pyruvate (TALP), Ham's F-10 plus 10% FCS, synthetic oviductal fluid (“SOF”), B2, CR1aa, medium and high potassium simplex medium (“KSOM”).

The reconstituted cell may also be activated by known methods. Such methods include, eg., culturing the reconstituted cell at sub-physiological temperature, in essence by applying a cold, or actually cool temperature shock to the reconstituted cell. This may be most conveniently done by culturing the reconstituted cell at room temperature, which is cold relative to the physiological temperature conditions to which embryos are normally exposed. Suitable oocyte activation methods are the subject of U.S. Pat. No. 5,496,720, to Susko-Parrish et al., herein incorporated by reference in its entirety.

The activated reconstituted cells may then be cultured in a suitable in vitro culture medium until the generation of cells and cell colonies. Culture media suitable for culturing and maturation of embryos are well known in the art. Examples of known media, which may be used for bovine embryo culture and maintenance, include Ham's F-10 plus 10% FCS, TCM-199 plus 10% FCS, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% FSC, 0.2 mM Na pyruvate and 50 μg/ml gentamicin sulphate. Any of the above may also involve co-culture with a variety of cell types such as granulosa cells, oviduct cells, BRL cells and uterine cells and STO cells.

Afterward, the cultured reconstituted cell or embryos are preferably washed and then placed in a suitable media, eg., TCM-199 medium containing 10% FCS contained in well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells.

In one embodiment, the feeder cells comprise mouse embryonic fibroblasts. Preparation of a suitable fibroblast feeder layers are well known in the art.

The reconstituted cells are cultured on the feeder layer until the reconstituted cells reach a size suitable for transferring to a recipient female, or for obtaining cells which may be used to produce cells or cell colonies. Preferably, these reconstituted cells will be cultured until at least about 2 to 400 cells, more preferably about 4 to 128 cells, and most preferably at least about 50 cells. The culturing will be effected under suitable conditions, i.e., about 39° C. and 5% CO₂, with the culture medium changed in order to optimise growth typically about every 2-5 days, preferably about every 3 days.

The methods for embryo transfer and recipient animal management in the present invention are standard procedures used in the embryo transfer industry. Synchronous transfers are important for success of the present invention, i.e., the stage of the nuclear transfer embryo is in synchrony with the estrus cycle of the recipient female. This advantage and how to maintain recipients are reviewed in Siedel, G. E., Jr. (“Critical review of embryo transfer procedures with cattle” in Fertilization and Embryonic Development in Vitro (1981) L. Mastroianni, Jr. and J. D. Biggers, ed., Plenum Press, New York, N.Y., page 323), the contents of which are hereby incorporated by reference.

Briefly, blastocysts may be transferred non-surgically or surgically into the uterus of a synchronized recipient. Other medium may also be employed using techniques and media well-known to those of ordinary skill in the art. In one procedure, cloned embryos are washed three times with fresh KSOM and cultured in KSOM with 0.1% BSA for 4 days and subsequently with 1% BSA for an additional 3 days, under 5% CO₂, 5% O₂ and 90% N₂ at 39° C. Embryo development is examined and graded by standard procedures known in the art. Cleavage rates are recorded on day 2 and cleaved embryos are cultured further for 7 days. On day seven, blastocyst development is recorded and one or two embryos, pending availability of embryos and/or animals, is transferred non-surgically into the uterus of each synchronized foster mother.

Foster mothers preferably are examined for pregnancy by rectal palpation or ultrasonography periodically, such as on days 40, 60, 90 and 120 of gestation. Careful observations and continuous ultrasound monitoring (monthly) preferably is made throughout pregnancy to evaluate embryonic loss at various stages of gestation. Any aborted fetuses should be harvested, if possible, for DNA typing to confirm clone status as well as routine pathological examinations.

The reconstituted cell, activated reconstituted cell, fetus and animal produced during the steps of such method, and cells, nuclei, and other cellular components which may be harvested therefrom, are also asserted as embodiments of the present invention. It is particularly preferred that the term animal produced be a viable animal.

The present invention can also be used to produce embryos, fetuses or offspring which can be used, for example, in cell, tissue and organ transplantation. By taking a fetal or adult cell from an animal and using it in the cloning procedure a variety of cells, tissues and possibly organs can be obtained from cloned fetuses as they develop through organogenesis. Cells, tissues, and organs can be isolated from cloned offspring as well. This process can provide a source of materials for many medical and veterinary therapies including cell and gene therapy. If the cells are transferred back into the animal in which the cells were derived, then immunological rejection is averted. Also, because many cell types can be isolated from these clones, other methodologies such as hematopoietic chimerism can be used to avoid immunological rejection among animals of the. same species.

Throughout the description and claims of this specification, the word “comprise” and variations of the word, such as “comprising” and “comprises”, is not intended to excluded other additives, components, integers or steps.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the use of mouse and porcine cells, it will be clearly understood that the findings herein are not limited to these-types of cells, but would be useful growing any type of cell from any animal.

### EXAMPLE 1

### Activation Of The Germline-Specific Oct4 Promoter in Murine Somatic Explant Cultures

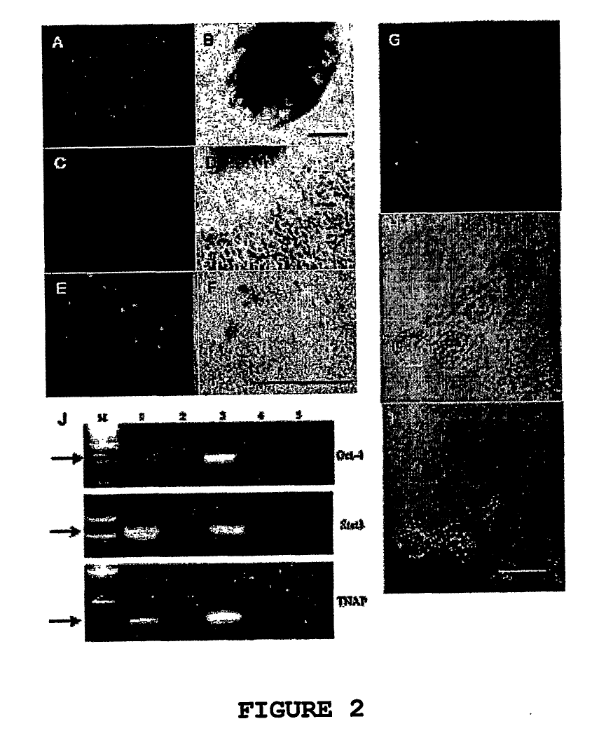

OG2-transgenic mice carrying the GFP reporter gene under transcriptional control of the exclusively germline-specific Oct-4 promoter, were employed for fetal explant cultures. Mesenchymal explants with an average size of <1 mm³ were isolated from fetuses of days 11.5, 13.5 and 14.5 p.c., pasted with recalcified microdrops of bovine plasma to cell culture dishes and cultured individually in Dulbecco's Modified Eagles Medium (DMEM) supplemented with 2 mM glutamine and 10% FCS as described infra. Specific care was taken to isolate explants from connective tissue of the neck and shoulder regions. Fluorescence microscopy using Zeiss Axiomat LSM and excitation wavelength of 488 nm was used to detect GFP. No GFP positive-cells were revealed in the initial explants and GFP expression could not be detected in the first outgrowths after 2 days (FIG. 2C, D). However, after 8 days of culture, GFP positive cells, indicative for the activation of the germ line-specific Oct4-eGFP marker cassette, were clearly detectable within the primary outgrowing cells (FIG. 2E, F). Subpassages of the outgrowing cells cultured in DMEM supplemented with 30% FCS maintained GFP-positive cells (FIG. 2G-I), however at relative low frequencies of 10⁻²-10⁻³.

The expression of the endogenous Oct4 gene was confirmed by RT-PCR detection of the corresponding mRNA in subpassages of the mesenchymal outgrowths (FIG. 2J). The genital ridges of the fetuses served as positive controls for the tissue-specificity of the Oct4-GFP cassette; the primordial germ cells showed massive expression of GFP for several days in culture (FIG. 2A, B), no outgrowing GFP positive cells could be detected.

### EXAMPLE 2

### Induction of Ap Expression and Loss of Contact-Inhibition

The isolation of Oct4 expressing cells from murine somatic explants raised the question whether similar cells could be obtained from livestock species. Mesenchymal explants of porcine fetuses (day 25 p.c.) were established and subpassaged once using standard culture protocols yielding morphologically homogenous cell cultures. Immunostaining showed uniform labelling with a vimentin specific and no labelling with a cytokeratin-specific antibody (data not shown), indicative for fibroblasts. RT-PCR with porcine Oct4 specific primers prove that cultures maintained in DMEM/30% FCS activated the germ line specific Oct4 gene (not shown).

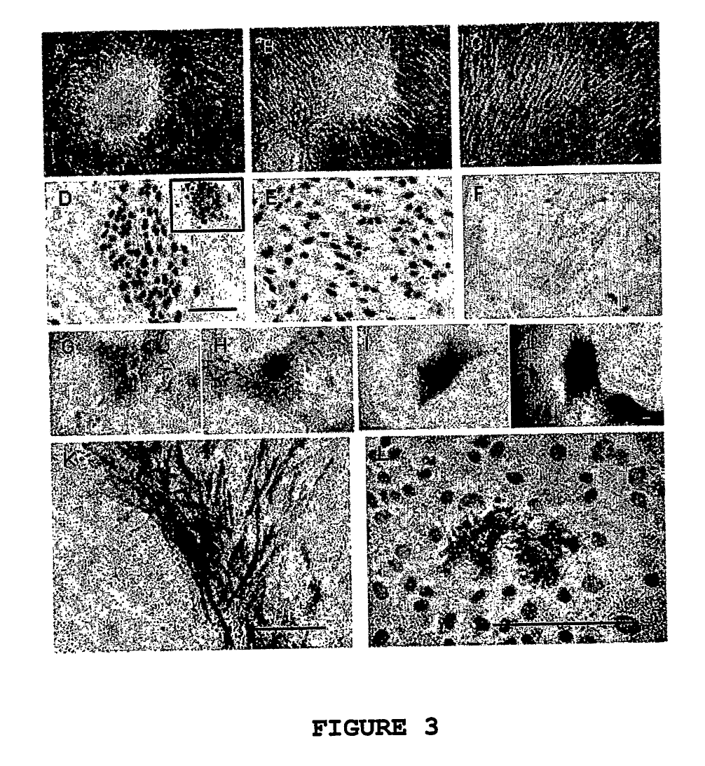

Upon change of the culture medium to high serum concentrations, ie. DMEM containing 30% FCS, the cultures did no longer show contact inhibition. After confluency was reached the growth of 3D-colonies became apparent (FIG. 3A, B). Only cells within the 3D-colonies proliferated as measured by BrdU incorporation, whereas the surrounding monolayer-forming cells were mitotically inactive (FIG. 3D). Control experiments with standard conditions showed. 80% BrdU-labelled cells during the proliferative phase of subconfluent and <2% BrdU positive cells in confluent cultures (FIG. 3E, F).

Staining. for endogenous alkaline phosphatase (AP) activity revealed a massive induction of AP-positive cells, which were nearly exclusively accumulated within the 3D-colonies (FIG. 3G-J). AP-positive cells showed a different morphology (FIG. 3K, L) from that of the common fibroblast-like type in that they displayed a dendritic morphology. If cells from the same batch were grown under standard culture conditions (with 10% FCS), the cultures became contact-inhibited, 3D-colony growth (FIG. 3C) did not occur and AP-positive cells were only rarely found at a frequency of 10⁻³-10⁻⁴ (Table 1). Approximately 6.7% of microwells seeded with ten cells from high serum cultures resulted in continuously growing cultures, suggesting that 1 out of 150 cells was able to initiate clonal growth. The effects of high serum supplementation were heat and trypsin sensitive (data not shown).

TABLE 1
HIGH SERUM INDUCTION OF AP-POSITIVE CELLS AND 3D-COLONIES
IN PORCINE AND MURINE CELL ISOLATES

| | | | | | high serum induced | |
|---|---|---|---|---|---|---|
| | | | | | AP-positive cells | |
| | | | | | (fold increase compared | 3D-colonies |
| | age of donors | tissue source | method | n | to standard cultures) | (no./6-well) |
| porcine | day 25-27 p.c. | mesoderm | try. | 2 | 250-850 | 100-290 |
| | day 25 p.c. | mesoderm | expl. | 12 | 100-1000 | 65-340 |
| | 0.5-1.5 years | ear biopsy | expl. | 3 | 2-10 | 0 |
| murine | day 11.5 p.c. | mesoderm | expl. | 3 | 8 | 3-5 |
| | day 13.5 p.c. | mesoderm | expl. | 1 | 3 | 0 |
| | day 14.5 p.c. | mesoderm | expl. | 3 | 11 | 3-5 |
| | 4 months | subdermal tissue | expl. | 1 | 1.5 | 0 |

Abbr.:
try., trypsinisation of pooled fetuses (n + 6-8);
expl., explant cultures from individual fetuses or adult subdermal tissues

Adult porcine fibroblasts (3 different origins, 0.5-1.5 y old donors) derived from subdermal tissue explants did not display 3D-colony growth (FIG. 3) when cultured in DMEM/30%FCS. However, the frequency of AP expressing cells was increased 2-10 fold in high serum cultures compared to control cultures (Table 1) while for fetal cells a 100-1000 fold increase had been calculated. Induction of 3D-colony growth and AP expression in murine cultures was at least one order of magnitude lower than in porcine cultures (Table 1).

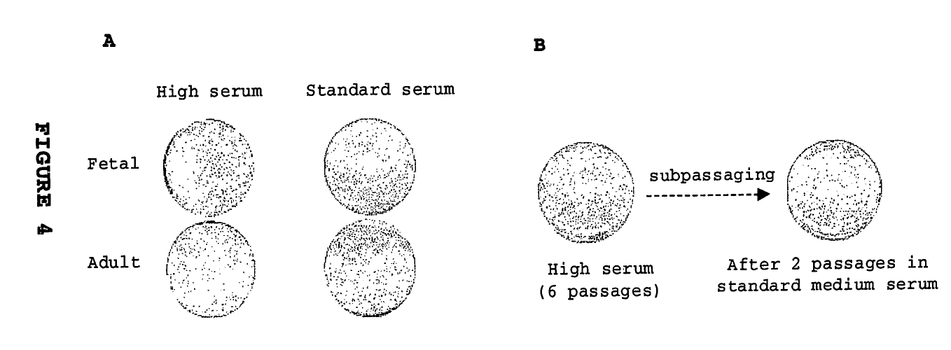

Apparently the altered phenotype of porcine fetal cultures was reversible. When high serum cultures were split and one part of the population was returned to standard medium, colony-growth ceased and AP-positive cells disappeared nearly completely within two passages, suggesting that induction and proliferation of MCTs are dependent upon high serum levels in culture (FIG. 4B).

### EXAMPLE 3

### Increased Proliferative Potential

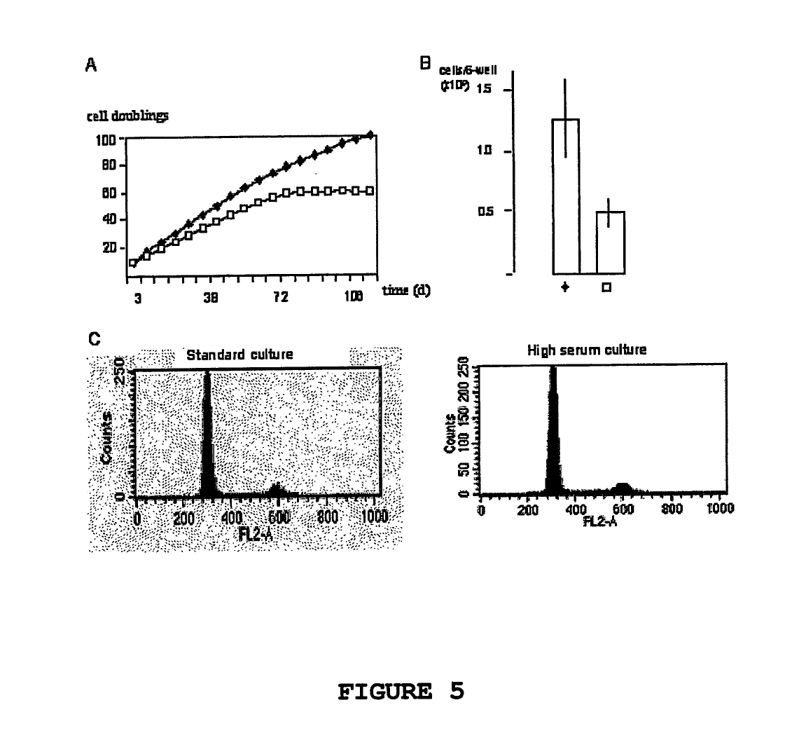

Culture medium supplemented with high-serum resulted in a dramatically altered growth curve (FIG. 5). Cultures maintained under high serum conditions grew continuously over a period of >120 days and exceeded more than 100 cell doublings without reaching a plateau phase (FIG. 5A). In contrast, standard cultures, ie. DMEM with 10% FCS, were compatible with only 50-60 cell doublings before mitotic activity ceased after app. 70 days. The total cell number of the DMEM/30% FCS cultures exceeded that of the standard cultures by a factor of up to 2.5 at each subpassage (FIG. 5B). The MCTs maintained a diploid status, as measured by fluorescence activated cell sorting (FIG. 5C) and metaphase spreads.

### EXAMPLE 4

### Formation of Spheroids and Anchorage-Independent Growth

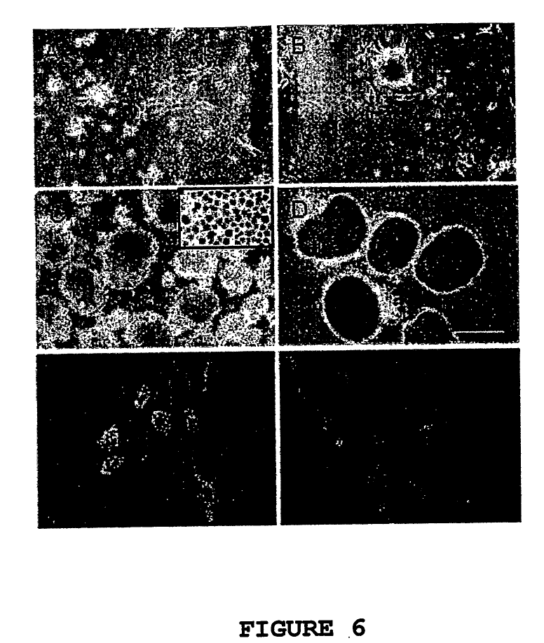

To investigate the growth potential of the colony forming fetal cells, 3D-colonies of 200-300 μm diameter were isolated and trypsinised to obtain single cells suspensions. Subsequently, 10⁴ cells were seeded into bacteriological culture dishes to prevent attachment. Supplementation of the culture medium (DMEM/30% FCS) with dexamethasone resulted in aggregation of small multicellular spheroids within 24 hours, which continued to grow up to a diameter of >400 μm after 10-15 days and contained nearly exclusively AP positive cells (FIG. 6C, D). Initially tiny aggregates were formed in culture medium supplemented with retinoic acid, which after 2-4 days attached to the surface and showed extensive outgrowth (FIG. 6B). In DMEM/30%FCS without supplement, small irregular aggregates consisting of only few cells (2-20) were detected. These cells did not expand and the majority apparently underwent cell death (FIG. 6A). If plated on gelatinised coverslips, dexamethasone-spheroids reattach and monolayer cells grew out. Immunohistology with a monoclonal antibody against vimentin showed no labelling, whereas control cultures kept in standard medium with 10% FCS were strongly positive (FIG. 6 E, F).

### EXAMPLE 5

### In Vivo Differentiation Potential by Injection of Mcts into Blastocysts

To determine the developmental potential, MCTs were injected into murine blastocysts, which were subsequently transferred to pseudopregnant recipients. MCTs of both sexes were isolated from double transgenic fetuses of OG2 and Rosa26 mouse strains. These cells carried the germline specific Oct-4 GFP and the ubiquitously active lacZ reporter gene constructs and thus allowed to distinguish them from the cells of the recipient blastocysts.

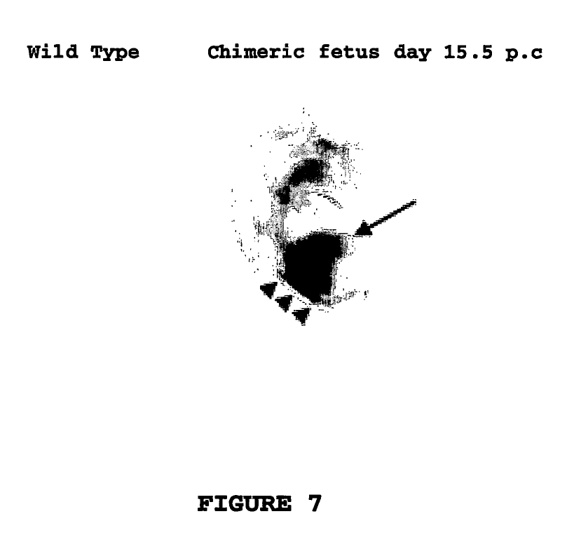

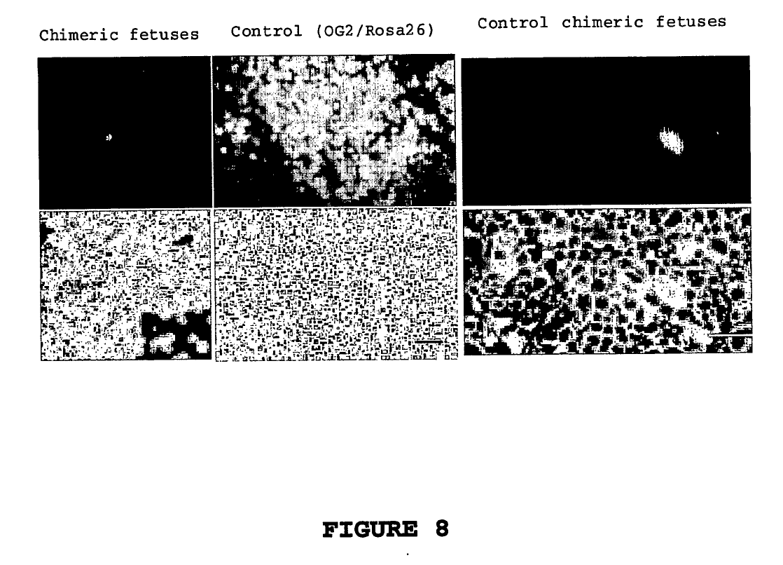

Day 13.5-15.5 fetuses derived from the injected blastocysts were isolated and analyzed for chimerism either by staining for lacZ activity or by fluorescence microscopy to identify GFP positive cells. Of a total of 19 analyzed fetuses, 7 contained progeny cells from the injected MCTs (Table 2). Chimerism was detected in mesenchymal organs, such as liver, muscle and tongue, but also in the genital ridges. FIG. 7 shows an example of a chimeric fetus with massive lacZ staining in liver, tongue and genital ridges, suggesting that at least parts of these organs were derived from the injected cells. Chimeric and wildtype fetuses were derived from embryo transfers that had been performed on the same day, were stained for LacZ activity in parallel and photographed on the same slide. It is unclear whether the apparent oversize of the chimeric fetus is related to the cell injection. The summarised data for the blastocyst transfer suggest that development of embryos after FSSC injection is compromised (Table 2). FIG. 8 shows the presence of GFP positive cells in the genital ridges of a male day 15.5 p.c. fetus, indicating that the descendants of the injected Rosa26/OG2 cells were capable of differentiation to primordial germ cells and could correctly migrate into the target organ. In total, 16 GFP-positive cells were counted in the squeeze preparation, and these cells behaved like primordial germ cells in that they floated within the ducts of the genital ridges. GFP positive cells were never found in other organs, such as heart, liver, brain or connective tissue.

TABLE 2
GENERATION OF CHIMERIC FETUSES BY INJECTION OF MCTs INTO
RECIPIENT BLASTOCYSTS

| | Transgenic | No.of blastocysts | Recovered | Chimeric | | |
|---|---|---|---|---|---|---|
| No. injected FSSCs | background | transferred | fetuses | fetuses | Assay | Positive cells found in: |
| 6-8 | Rosa26 | 57 | 4 | 0 of 4 | LacZ | none |
| 10-15 | Rosa26 | 6 | 1 | 1 of 1 | LacZ: | liver, genital ridge, tongue |
| 2-5 | Rosa26/OG2 | 24 | 5 | 3 of 5 | LacZ: | mesoderm, sev. organs, low chimerism |
| 6-8 | Rosa26/OG2 | 49 | 9 | 2 of 7 | LacZ: | mesoderm, sev, organs, low chimerism |
| | | | | 1 of 2 | OG2: | gential ridge |
| 10-15 | Rosa26/OG2 | 20 | 0 | — | | n.a. |
| control blastocysts | wt | 29 | 14 | 0 of 9 | LacZ: | some background in spinal cord |
| w/o cell injection | | | | 0 of 5 | OG2: | — |

### DISCUSSION

The present invention demonstrates the presence of tissue-specific progenitor cells or stem cell-like cells (MCTS) in fetal mesenchymal tissue cultures of rodents and livestock species that can be specifically enriched by the methods disclosed herein. MCT cells are characterised by extended proliferative capacity, altered morphology, de novo expression of the stem cell markers Oct4, Stat3 and AP, as well as contact- and anchorage-independent growth.

The explant culture technique employing higher than normal serum levels seems to be essential for an initially stimulation of the MCT proliferation. Standard culture using low serum levels of 10% or less are associated with a progressive loss of MCTS.

Transcriptional activity of the Oct4 promoter in MCTs indicates that these cells have characteristics of stem cells. Oct4 controls the expression of several genes including Fgf4, Rex-1, Sox-2, OPN, hCG, Utf-1 and INFt. Variation in the level of Oct-4 expression by as little as 30% has been shown to maintain cells either in the totipotent state or to drive embryonic stem cells into differentiation.

Chimeric fetuses, obtained by injection of murine MCTs into recipient blastocysts, showed that the MCTs were able to contribute to various mesenchymal organs and in particular the genital ridges. Genital ridges showed contribution of MCTs to the primordial germ cells, as some albeit few cells expressed GFP fluorescence driven by the germ line specific Oct-4 promoter, indicating that germ line transmission might be possible. The finding that GFP positive cells were not found outside of the genital ridges indicates that the Oct-4 marker was correctly activated in cells committed to the germ line. It also suggests that at least some of the MCTs descendants were capable to migrate into the genital ridge. The relatively low percentage of chimerism might be due to the fact, that the cells used for blastocyst injection were not preselected for Oct-4-GFP expression.

Preferentially, chimerism was found in liver, muscle and tongue. No chimerism was detected in heart and brain, two organs, which showed a high rate of spontaneous cell fusions in a recent study. However, we cannot fully exclude the possibility that fusion with differentiated cells might have contributed to the observed chimerism.

Two remarkable characteristics of MCTs are 3D-colony growth and the ability to grow in suspension. Our data provides convincing evidence that unlike many cell lines derived from tumours or cells transformed by oncogenic agents, the MCT subpopulation does not result from spontaneous immortalisation or transformation. MCTs do not exhibit a crisis followed by clonal outgrowth and chromosomal abnormalities or aneuplodies, and show reversibility of the altered growth characteristics after exposure to standard cell culture conditions.

### EXPERIMENTAL PROTOCOL

### Cell Culture Of Fetal And Adult Fibroblasts

Primary fibroblasts were prepared by enzymatic isolation of eviscerated fetuses or by mesenchymal explant (<1 mm³) cultures of connective tissue pasted to the dish surface by employing recalcified microdrops of bovine plasma and maintained in Dulbecco's Modified Eagles Medium (DMEM) medium supplemented with 1 mM glutamine, 1%.non-essential amino acids 1% vitamin solution, 0.1 mM mercaptoethanol, 100 U/ml penicillin, 100 mg/ml streptomycin (all from Sigma, Deisenhofen, Germany), containing 10% FCS from selected batches (Gibco, Karlsruhe, Germany, batch numbers 40G321K, 40G2810K) and incubated in a humidified 95% air/5% C0₂ atmosphere at 37° C. (Keus et al., 2000, Biol. Reprod., 62: 412-419; Keus et al., 2002, Cloning Stem Cells, 4: 147-165). Outgrowing cells were trypsinised and subpassaged once prior to cryoconservation. For high serum culture the serum content of the standard medium was increased to 30% FCS. For suspension culture, colonies were selectively isolated and completely dissociated in a trypsin solution, then 10⁴ cells were seeded into bacteriological dishes (35 mm). Every second day 50% of the medium was replaced with new medium. To determine the maximal replicative limit, cultures were serially subpassaged and 12.5×10³ cells were seeded per cm² in 6-well-dishes, trysinised after 5-7 days, counted and reseeded. The number of accumulated population doublings per passage was determined using the equation, PD =log (A/B)/log2, in which A is the number of collected cells and B is the number of plated cells. Murine fibroblasts were obtained from day 11.5-14.5 fetuses or adult animals of OG2 mice (Chang et al., 2002, Proc. Natl. Acad. Sci. USA; 99:12877-12882) (homozygous for a Oct4-GFP transgene) or from double transgenic fetuses of crosses of OG2 with Rosa26 mice. Confocal microscopy was applied to detect GFP using a Zeiss Axiomat LSM and an excitation wavelength of 488 nm. ES cells (wild type GS1 129/Sv) were cultured as described previously (Gotz et al., 1998, Proc Natl Acad Sci USA; 95:12370-12375).

### RT-PCR Detection of OCT4 and eGFP mRNAs

In brief, total RNA was isolated from cells grown in 6-well dishes and reverse transcribed into cDNA using random hexamers as primers. Murine Oct4 and GFP cDNAs were amplified by PCR with the following primers and conditions:

5′-GGC GTT CTC TTT GGA AAG GTG TTC,
and
5′-CTC GAA CCA CAT CC TTC TCT

(35 cycles, annealing temperature 57° C.) for the murine Oct4:

5′-TGA CCC TGA AGT TCA TCT GC
and
5′-TGA AGT TCA CCT TGA TGC CG

(35 cycles) for GFP. Porcine Oct4 was amplified with:

5′-AGGTGTTCAGCCAAACGACC
and
5′-TGATCGTTTGCCCTTCTGGC

primers (AJ251914) and 36 cycles. The PCR reactions were performed in 20 μl volumes, consisting of 20 mM Tris.HCl (pH 8.4), 50 mM KCl, 1.5 mM MgCl₂, 200 μM dNTPs, 1 μM of specific primer pairs and 0.5 units of Taq DNA polymerase (Gibco).

### Measurement of Cell Proliferation By Brdu Incorporation

DNA synthesis was measured by 5-bromo-2′deoxy-uridine (BrdU) incorporation as described in Keus et al. (2002, Cloning Stem Cells, 4:231-243). Incorporated BrdU was detected by a chromogenic immunoassay employing an anti-BrdU antibody conjugated with alkaline phosphatase.

### Immunohistology

Cells grown on gelatinised coverslips, were fixed in cold 80% methanol. The following monoclonal antibody dilutions were used: anti-vimentin (AMF-17b, 1:200) (Developmental Studies Hybridoma Bank, Iowa) and anti-cytokeratin (peptide 17, 1:100, Sigma). A rhodamine-labelled secondary anti-mouse antibody (1:2000, Molecular Probes, N L) was used. In some cases the nuclei were counterstained with 1 mM Hoechst 33342 (Keus et al, 1995, J. Cell Biol., 130: 949-957). The samples were examined with an Olympus BX60 microscope equipped with phase-contrast and epifluorescence optics, using band-pass rhodamine and Hoechst filter sets.

### Staining of Endogenous Alkaline Phosphatase Activity

Cell cultures were washed with PBS, fixed in 3.7% paraformaldehyde for 15 minutes, washed in PBS and then incubated in a solution containing 25 mM TrisHC pH 9.0, 4 mM MgCl₂, 0.4 mg/ Na-α-naphtylphosphate, 1 mg/ml Fast Red TR (Sigma) and 0.05% Triton X-100 for 60 minutes.

### Chimera Generation by Mcts Injection into Host Blastocysts

Rosa26 homozygous mice were obtained from Jackson Laboratory (NY) and mated with homozygous OG2 animals to generate double-transgenic fetuses carrying both marker genes, which were used to isolate MCTs. Between day 11.5 and day 15.5 fetuses were isolated and employed for fetal cell cultures using the explant method described supra.

For blastocyst injections, 6-10 week old female CD2F1 mice were superovulated with 10 U PMSG at noon on day -2, followed by 10 U hCG on day 0 and were then mated with CD2F1 males. The next day females were checked for plug formation. At day 3.5 females were sacrificed, and the uterine tracts were isolated and flushed with PBS containing 1% albumin. Blastocysts were isolated and incubated in 1% albumin at 37° C. Single blastocysts were transferred into a micromanipulation unit (Zeiss) and fixed with a holding pipette. On average 2-15 double transgenic cells (OG2/Rosa26) were injected into the blastocoel by the aid of a microcapillary. In total, 8-10 blastocysts were transferred into the uteri of day 2.5 or day 3.5 pseudopregnant NMRI females that had been obtained by matings of NMRI females with vasectomised males. Fetuses were recovered at day 10.5-15.5 and either stained for lacZ positive cells (Friedrich & Soriano, 1991, Genes Dev., 5, 1513-1523) as whole mounts, or dissected and screened for GFP expression in genital ridges and other organs.

1. A method for selective culturing of primary cell cultures comprising culturing tissue biopsies in the presence of at least 25% serum relative to the amount of culture medium.

2. A method according to claim 1, wherein the serum is between about 25% to about 70%.

3. A method according to claim 1, wherein the serum is between about 30% to about 50%.

4. A method according to claim 1, wherein the serum is between about 30%.

5. A tissue-culture media composition for the selective culturing of primary cell cultures comprising about 30% serum and about 70% culture medium.

6. A tissue-culture media composition according to claim 5, wherein the culture medium is selected from the group consisting of Synthetic Oviductal Fluid (SOF), Modified Eagle's Medium (MEM), Dulbecco's Modified Eagle's Medium (DMEM), RPMI 1640, F-12, IMDM, Alpha Medium and McCoy's Medium.

7. A tissue-culture media composition according to claim 5 or claim 6, wherein the serum is selected from the group consisting of allogeneic serum, autologous serum AND xenogeneic serum.

8. A tissue-culture media composition according to claim 5 or claim 6, wherein the serum is heat-inactivated autologous serum.

9. A tissue-culture media composition according to any one of claims 5 to 8, further comprising growth factors, co-factors, salts or antibiotics.

10. A method for selective culturing of primary cell cultures comprising:
(i) obtaining a tissue biopsy from an animal;
(ii) culturing said tissue biopsy in tissue culture medium comprising at least 25% serum; and
(iii) replacing about 50% of the culture medium including serum about every 48 hours.

11. A method according to claim 10, wherein the tissue biopsies are cultured in the presence of a feeder cell layer.

12. A method according to claim 11, wherein the feeder cell layer comprises cultured autologous cells.

13. A method according to any one of claims 10 to 12, wherein the tissue biopsies are obtained from a mammalian animal.

14. A method according to claim 13, wherein the mammalian animal is selected from the group consisting of platypus, echidna, kangaroo, wallaby, shrews, moles, hedgehogs, tree shrews, elephant shrews, bats, primates (including chimpanzees, gorillas, orang-utans, humans), edentates, sloths, armadillos, anteaters, pangolins, rabbits, picas, rodents, whales, dolphins, porpoises, carnivores, aardvark, elephants, hyraxes, dugongs, manatees, horses, rhinos, tapirs, antelope, giraffe, cows or bulls, bison, buffalo, sheep, big-horn sheep, horses, ponies, donkeys, mule, deer, elk, caribou, goat, water buffalo, camels, llama, alpaca, pigs and hippos.

15. A method according to claim 13, wherein the tissue biopsies are isolated from an ungulate selected from the group consisting of domestic or wild bovid, ovid, cervid, suid, equid and camelid.

16. A method according to claim 13, wherein the tissue biopsies are isolated from a human subject.

17. A method according to any one of claims 13 to 16, wherein the tissue biopsies are obtained from an organ selected from the group consisting of skin, lung, pancreas, liver, stomach, intestine, heart, reproductive organs, bladder, kidney urethra and other urinary organs.

18. A method according to claim 17, wherein the tissue biopsies are obtained from fetal tissue.

19. A method according to claim 17, wherein the tissue biopsies are obtained from adult tissue.

20. An isolated tissue-specific progenitor cell or stem cell-like cell obtained by a method according to any one of claims 1 to 19.

21. An isolated tissue-specific progenitor cell according to claim 20, wherein the cell is a mesenchymal connective tissue-derived stem cell.

22. An isolated mesenchymal connective tissue-derived stem cell.

23. An isolated mesenchymal connective tissue-derived stem cell according to claim 22, wherein the cell has the capacity to be induced to differentiate to form at least one differentiated cell type of mesodermal, ectodermal and endodermal origin.

24. A cell according to claim 22 or claim 23, wherein said cell is derived from a non-embryonic organ or tissue of a mammal.

25. A cell according to any one of claims 22 to 24, wherein the cell has the capacity to be induced to differentiate to form cells selected from the group consisting of osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal and oligodendrocyte cell type.

26. A cell according to claim 24, wherein the organ or tissue is selected from the group consisting of bone marrow, muscle, brain, umbilical cord blood and placenta.

27. A cell according to any one of claims 24 to 27, wherein the mammal is a human.

28. A cell according to any one of claims 23 to 27, wherein differentiation is induced in vivo or ex vivo.

29. A cell according to any one of claims 22 to 28, wherein the cell constitutively expresses oct4 and high levels of telomerase.

30. An isolated mesenchymal connective tissue-derived stem cell as deposited under the Budapest Treaty at the Deutsche Samnlung Von Mikroorganismen und Zellkulturen GmbH (DSMZ), Germany on September 2004, under accession number #12345.

31. A method of creating a normal non-human animal comprising the steps of: (a) introducing a mesenchymal connective tissue-derived stem cell into a blastocyst; (b) implanting the blastocyst of (a) into a surrogate mother; and (c) allowing the pups to develop and be born.

32. A method according to claim 31, wherein the normal non-human animal is a chimeric animal.

33. A composition comprising a population of a mesenchymal connective tissue-derived stem cell and a culture medium, wherein the culture medium expands the mesenchymal connective tissue-derived stem cells.

34. A composition according to claim 33, wherein the culture medium comprises epidermal growth factor (EGF) and platelet derived growth factor (PDGF).

35. A composition according to claim 34, wherein the culture medium further comprises leukemia inhibitory factor (LIF).

36. A composition comprising a population of fully or partially purified a mesenchymal connective tissue-derived stem cell progeny.

37. A composition according to claim 36, wherein the progeny have the capacity to be further differentiated.

38. A composition according to claim 36, wherein the progeny have the capacity to terminally differentiate.

39. A composition according to claim 36, wherein the progeny are of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

40. A method for isolating and propagating a mesenchymal connective tissue-derived stem cell comprising the steps of: (a) obtaining tissue from a mammal; (b) establishing a population of adherent cells; (c) recovering said mesenchymal connective tissue-derived stem cells; and (d) culturing mesenchymal connective tissue-derived stem cells under expansion conditions to produce an expanded cell population.

41. An expanded cell population obtained by a method according to claim 40.

42. A method for differentiating mesenchymal connective tissue-derived stem cells ex vivo comprising the steps of (a) obtaining tissue from a mammal; (b) establishing a population of adherent cells; (c) recovering said mesenchymal connective tissue-derived stem cells; (d) culturing mesenchymal connective tissue-derived stem cells under expansion conditions to produce an expanded cell population and (e) culturing the propagated cells in the presence of desired differentiation factors.

43. A method according to claim 42, wherein the differentiation factors are selected from the group consisting of basic fibroblast growth factor (bFGF); vascular endothelial growth factor VEGF); dimethylsulfoxide (DMSO) and isoproterenol; and, fibroblast growth factor4 (FGF4) and hepatocyte growth factor (HGF).

44. A method according to claim 42, wherein the differentiated cell obtained by said method is ectoderm, mesoderm or endoderm.

45. A method according to claim 42, wherein the differentiated cell obtained by said method is of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

46. A method for differentiating a mesenchymal connective tissue-derived stem cell in vivo comprising the steps of (a) obtaining tissue from a mammal; (b) establishing a population of adherent cells; (c) recovering said mesenchymal connective tissue-derived stem cells; (d) culturing mesenchymal connective tissue-derived stem cells under expansion conditions to produce an expanded cell population and (e) administering the expanded cell population to a mammalian host, wherein said cell population is engrafted and differentiated in vivo in tissue specific cells, such that the function of a cell or organ, defective due to injury, genetic disease, acquired disease or iatrogenic treatments, is augmented, reconstituted or provided for the first time.

47. A method according to claim 46, wherein the tissue specific cells are of the osteoblast, chondrocyte, adipocyte, fibroblast, marrow stroma, skeletal muscle, smooth muscle, cardiac muscle, occular, endothelial, epithelial, hepatic, pancreatic, hematopoietic, glial, neuronal or oligodendrocyte cell type.

48. A method according to claim 46 or claim 47, wherein the mesenchymal connective tissue-derived stem cell undergoes self-renewal in vivo.

49. A method according to any one of claims 46 to 48, wherein cells are administered in conjunction with a pharmaceutically acceptable matrix.

50. A method according to claim 49, wherein the matrix is biodegradable.

51. A method according to any one of claims 46 to 50, wherein administration is via localized injection, systemic injection, parenteral administration, oral administration, or intrauterine injection into an embryo.

52. A method according to claim 51, wherein localized injection comprises catheter administration.

53. A method according to any one of claims 46 to 52, wherein the disease is selected from the group consisting of cancer, cardiovascular disease, metabolic disease, liver disease, diabetes, hepatitis, hemophilia, degenerative or traumatic neurological conditions, autoimmune disease, genetic deficiency, connective tissue disorders, anemia, infectious disease and transplant rejection.

54. A differentiated cell obtained by a method according to any one of claims 46 to 53.

55. A method of treatment comprising administering to an animal in need thereof a therapeutically effective amount of a cell according to claim 54.

56. A method according to claim 55, wherein no teratomas are formed in the animal.

57. A method of treatment comprising administering to an animal in need thereof a therapeutically effective amount of mesenchymal connective tissue-derived stem cells or their progeny.

58. A method according to claim 57, wherein reduced or no pretreatment of the animal is required.

59. A method according to claim 58, wherein pretreatment comprises myeloablation via irradiation or chemotherapy.

60. A method according to claim 57, wherein post immunosuppressive treatment of the patient is reduced compared with traditional pharmacological doses.

61. A method according to any one of claims 57 to 60, wherein the progeny have the capacity to be further differentiated.

62. A method according to claim 61, wherein the progeny are terminally differentiated.

63. A method according to any one of claims 57 to 62, wherein the mesenchymal connective tissue-derived stem cells or their progeny are administered via localized injection, systemic injection, parenteral administration, oral administration, or intrauterine injection into an embryo.

64. A method according to claim 63, wherein localized injection comprises catheter administration.

65. A method according to any one of claims 57 to 64, wherein cells are administered in conjunction with a pharmaceutically acceptable matrix.

66. A method according to claim 65, wherein the matrix is biodegradable.

67. A method according to any one of claims 57 to 66, wherein the mesenchymal connective tissue-derived stem cells or their progeny alter the immune system to resist viral, bacterial or fungal infection.

68. A method according to any one of claims 57 to 66, wherein the mesenchymal connective tissue-derived stem cells or their progeny augment, reconstitute or provide for the first time the function of a cell or organ defective due to injury, genetic disease, acquired disease or iatrogenic treatments.

69. A method according to claim 68, wherein the organ is selected from the group consisting of bone marrow, blood, spleen, liver, lung, intestinal tract, eye, brain, immune system, circulatory system, bone, connective tissue, muscle, heart, blood vessels, pancreas, central nervous system, peripheral nervous system, kidney, bladder, skin, epithelial appendages, breast-mammary glands, fat tissue, and mucosal surfaces including oral esophageal, vaginal and anal.

70. A method according to any one of claims 57 to 69, wherein the mesenchymal connective tissue-derived stem cells or their progeny undergo self-renewal in vivo.

71. A method according to claim 68, wherein the disease is selected from the group consisting of cancer, cardiovascular disease, metabolic disease, liver disease, diabetes, hepatitis, hemophilia, degenerative or traumatic neurological conditions, autoimmune disease, genetic deficiency, connective tissue disorders, anemia, infectious disease and transplant rejection.

72. A method according to any one of claims 57 to 71, wherein the progeny are differentiated ex vivo or in vivo.

73. A method according to claim 72, wherein the progeny are selected from the group consisting of osteoblasts, chondrocytes, adipocytes, fibroblasts, marrow stroma, skeletal muscle, smooth muscle, cardiac muscle, occular endothelial, epithelial, hepatic, pancreatic, hematopoietic, glial, neuronal and oligodendrocytes.

74. A method according to any one of claims 57 to 73, wherein the mesenchymal connective tissue-derived stem cells or their progeny home to one or more organs in the animal and are engrafted therein such that the function of a cell or organ, defective due to injury, genetic disease, acquired disease or iatrogenic treatments, is augmented, reconstituted or provided for the first time.

75. A method according to claim 74, wherein the disease is selected from the group consisting of cancer, cardiovascular disease, metabolic disease, liver disease, diabetes, hepatitis, hemophilia, degenerative or traumatic neurological conditions, autoimmune disease, genetic deficiency, connective tissue disorders, anemia, infectious disease and transplant rejection.

76. A method according to claim 74, wherein the injury is ischemia or inflammation.

77. A method according to claim 74, wherein the organ is selected from the group consisting of bone marrow, blood, spleen, liver, lung, intestinal tract, eye, brain, immune system, circulatory system, bone, connective tissue, muscle, heart, blood vessels, pancreas, central nervous system, peripheral nervous system, kidney, bladder, skin, epithelial appendages, breast-mammary glands, fat tissue, and mucosal surfaces including oral esophageal, vaginal and anal.

78. A method according to any one of claims 57 to 77, wherein the mesenchymal connective tissue-derived stem cells or their progeny are genetically transformed to deliver a therapeutic agent.

79. A therapeutic composition comprising mesenchymal connective tissue-derived stem cells and a pharmaceutically acceptable carrier, wherein the mesenchymal connective tissue-derived stem cells are present in an amount effective to produce tissue selected from the group consisting of bone marrow, blood, spleen, liver, lung, intestinal tract, eye, brain, immune system, bone, connective tissue, muscle, heart, blood vessels, pancreas, central nervous system, kidney, bladder, skin, epithelial appendages, breast-mammary glands, fat tissue, and mucosal surfaces including oral esophageal, vaginal and anal.

80. A therapeutic method for restoring organ, tissue or cellular function to a mammalian animal in need thereof comprising the steps of: (a) removing mesenchymal connective tissue-derived stem cells from a mammalian donor; (b) expanding a mesenchymal connective tissue-derived stem cells to form an expanded population of undifferentiated cells; and (c) administering the expanded cells to the mammalian animal, wherein organ, tissue or cellular function is restored.

81. A method according to claim 80, wherein the function is enzymatic.

82. A method according to claim 80, wherein the function is genetic.

83. A method according to claim 80, wherein the mammalian donor is the patient.

84. A method according to any one of claims 80 to 83, wherein the organ, tissue or cell is selected from the group consisting of bone marrow, blood, spleen, liver, lung, intestinal tract, eye, brain, immune system, bone, connective tissue, muscle, heart, blood vessels, pancreas, central nervous system, peripheral nervous system, kidney, bladder, skin, epithelial appendages, breast-mammary glands, fat tissue, and mucosal surfaces including oral esophageal, vaginal and anal.

85. A method of inhibiting the rejection of a heterologous mesenchymal connective tissue-derived stem cells transplanted into a patient comprising the steps of: (a) introducing into the mesenchymal connective tissue-derived stem cells, ex vivo, a nucleic acid sequence encoding the recipient's MHC antigens operably linked to a promotor, wherein the MHC antigens are expressed by the mesenchymal connective tissue-derived stem cells; and (b) transplanting the mesenchymal connective tissue-derived stem cells into the patient, wherein MHC antigens are expressed at a level sufficient to inhibit the rejection of the transplanted mesenchymal connective tissue-derived stem cells.

86. A method according to claim 85, wherein the patient is of the same species or another mammalian species as the donor of the mesenchymal connective tissue-derived stem cells.

87. A method according to claim 85, wherein the mesenchymal connective tissue-derived stem cells are transplanted into the patient via localized injection, systemic injection, parenteral administration, oral administration, or intrauterine injection into an embryo.

88. A method according to claim 87, wherein localized injection comprises catheter administration.

89. A method according to any one of claims 85 to 88, wherein cells are transplanted in conjunction with a pharmaceutically acceptable matrix.

90. A method according to claim 89, wherein the matrix is biodegradable.

91. A method of nuclear transfer comprising the step of transferring a mesenchymal connective tissue-derived stem cell or nuclei isolated from a mesenchymal connective tissue-derived stem cell into an enucleated oocyte.

92. A method for producing a genetically engineered or transgenic non-human mammal comprising:
(i) inserting, removing or modifying a desired gene in a mesenchymal connective tissue-derived stem cell from a non-human mammal or nuclei isolated from a mesenchymal connective tissue-derived stem cell isolated from a non-human mammal; and
(ii) transferring the a mesenchymal connective tissue-derived stem cell or nuclei into an enucleated oocyte.

93. A method for producing a genetically engineered or transgenic non-human mammal comprising:
(i) inserting, removing or modifying a desired gene or genes in a mesenchymal connective tissue-derived stem cell from a non-human mammal or nuclei isolated from a mesenchymal connective tissue-derived stem cell isolated from a non-human mammal; and
(ii) inserting a mesenchymal connective tissue-derived stem cell or nuclei into an enucleated oocyte under conditions suitable for the formation of a reconstituted cell;
(iii) activating the reconstituted cell to form an embryo;
(vi) culturing said embryo until greater than the 2-cell developmental stage; and
(v) transferring said cultured embryo to a host mammal such that the embryo develops into a transgenic fetus.

94. A method for cloning a non-human mammal comprising:
(i) inserting a mesenchymal connective tissue-derived stem cell from a non-human mammal or nuclei isolated from a mesenchymal connective tissue-derived stem cell isolated from a non-human mammal into an enucleated mammalian oocyte, under conditions suitable for the formation of a reconstituted cell;
(ii) activating the reconstituted cell to form an embryo;
(iii) culturing said embryo until greater than the 2-cell developmental stage; and
(iv) transferring said cultured embryo to a host mammal such that the embryo develops into a fetus.

95. A method according to any one of claims 91 to 94, wherein the oocytes are isolated from either oviducts and/or ovaries of live animals.

96. A method according to any one of claims 91 to 95, wherein the oocytes are enucleated oocytes and zona pellucida-free.

97. A method according to claim 96, wherein the step of removing the zona pellucida is by a method selected from the group consisting of physical manipulation, chemical treatment and enzymatic digestion.

98. A method according to claim 96, wherein the step of removing the zona pellucida is by enzymatic digestion.

99. A method according to claim 98, wherein the enzyme used to digest the zona pellucida is a protease, a pronase or a combination thereof.

100. A method according to claim 99, wherein the enzyme is a pronase.

101. A method according to claim 99, wherein the enzyme is a pronase.

102. A method according to claim 99, wherein the enzyme is a pronase.

103. A method according to claim 100, wherein the pronase is used at a concentration between 0.1 to 5%.

104. A method according to claim 100, wherein the pronase is used at a concentration between 0.25% to 2%.

105. A method according to claim 100, wherein the pronase is used at a concentration of about 0.5%.


SEQUENCE LISTING (6 sequences)

SEQ ID NO: 1 - DNA, 24 bp, Artificial
  synthetic
ggcgttctctttggaaaggtgttc

SEQ ID NO: 2 - DNA, 20 bp, Artificial
  synthetic
ctcgaaccacatccttctct

SEQ ID NO: 3 - DNA, 20 bp, Artificial
  synthetic
tgaccctgaagttcatctgc

SEQ ID NO: 4 - DNA, 20 bp, Artificial
  synthetic
tgaagttcaccttgatgccg

SEQ ID NO: 5 - DNA, 20 bp, Artificial
  synthetic
aggtgttcagccaaacgacc

SEQ ID NO: 6 - DNA, 20 bp, Artificial
  synthetic
tgatcgtttgcccttctggc